SEQUENCE LISTING (2 sequences)

SEQ ID NO: 1 - RNA, 1543 nt, Dolosigranulum pigrum
  source 1..1543: mol_type = rRNA
atgagagtttgatcttggctcaggacgaacgctggcggcatgcctaatacatgcaagtcgaacgatgatatcactgcttgcagtgattgattagtggcgaacgggtgagtaacacgtgaggaacttgcccatgagcgggggacaacattcggaaacggatgctaataccccataggtggattggtcgcatgacgaattcattaaaggtggctttgctaccactcatggatagcctcgcggcgtattagctagttggtaaggtaatggcttaccaaggcagtgatacgtagccgacttgagagggtgatcggccacactgggactgagacacggcccagactcctacgggaggcagcagtagggaatcttccacaatgggtgcaaacctgatggagcaatgccgcgtgagtgaagaaggtcttcggatcgtaaagctctgttgttagagaagaacacgtgctaggtaactactagcgccttgacggtatctaaccagaaagtcacggctaactacgtgccagcagccgcggtaatacgtaggtgacaagcgttgtccggatttattgggcgtaaagggagcgcaggcggtctgtttagtctaatgtgaaagcccacggcttaaccgtggaacggcattggaaactgacagacttgaatgtagaagaggaaaatggaattccaagtgtagcggtggaatgcgtagatatttggaggaacaccagtggcgaaggcgattttctggtctaacattgacgctgaggctcgaaagcgtggggagcgaacaggattagataccctggtagtccacgccgtaaacgatgagtgctaagtgttgggggctttccggccctcggtgctggagctaacgtattaagcactccgcctggggattacgaccgcaaggttgaaactcaaaagaattgacggggacccgcacaagcggtggagcatgtggtttaattcgaagcaacgcgaagaaccttaccagctcttgacatcttctgacaactctagagatagagcgttcccttcggggacagaatgacaggtggtgcatggttgtcgtcagctcgtgtcgtgagatgttgggttaagtcccgtaacgagcgcaacccttgttgttagttgccagcattaagttgggcactttaacgagactgccggtgataaaccggaggaaggcggggatgacgtcaaatcatcatgccccttatgagctgggctacacacgtgctacaatggatggtacaacgagcagcgatcccgcaagggctagctaatctttcaaagccattctcagttcggattgcactctgcaactcgagtgcatgaagtcggaatcgctagtaatcgcgaatcagaatgtcgcggtgaatccgttcccgggtcttgtacacaccgcccgtcacaccacgagagtttgaaacacccgaagtcggccggccaaccctatgggaggcagccgtcgaaggtgggttgaatgattggggtgaagtcgtaacaaggtagccgtatcggaaggtgcggctggatcacctccttt

SEQ ID NO: 2 - RNA, 1383 nt, Dolosigranulum pigrum
  source 1..1383: mol_type = rRNA
tcnctgcttgcagtgantgattagtggcgaacgggtgagtaacacgtgaggaacttgcccatgagcgggggacaacattcggaaacggatgctaataccccataggtggattggtcgcatgacgaattcattaaaggtggctttgctaccactcatggatagcctcgcggcgtattagctagttggtaaggtaatggcttaccaaggcagtgatacgtagccgacttgagagggtgatcggccacactgggactgagacacggcccagactcctacgggaggcagcagtagggaatcttccacaatgggtgcaaacctgatggagcaatgccgcgtgagtgaagaaggtcttcggatcgtaaagctctgttgttagagaagaacacgtgctaggtaactactagcgccttgacggtatctaaccagaaagtcacggctaactacgtgccagcagccgcggtaatacgtaggtgacaagcgttgtccggatttattgggcgtaaagggagcgcaggcggtctgtttagtctaatgtgaaagcccacggcttaaccgtggaacggcattggaaactgacagacttgaatgtagaagaggaaaaatggaattccaagtgtagcggtggaatgcgtagatatttggaggaacaccagtggcgaaggcgattttctggtctaacattgacgctgaggctcgaaagcgtggggagcgaacaggattagataccctggtagtccacgccgtaaacgatgagtgctaagtgttgggggctttccggccctcggtgctggagctaacgtattaagcactccgcctggggattacgaccgcaaggttgaaactcaaaagaattgacggggacccgcacaagcggtggagcatgtggtttaattcgaagcaacgcgaagaaccttaccagctcttgacatcttctgacaactctagagatagagcgttcccttcggggacagaatgacaggtggtgcatggttgtcgtcagctcgtgtcgtgagatgttgggttaagtcccgtaacgagcgcaacccttgttgttagttgccagcattaagttgggcactttaacgagactgccggtgataaaccggaggaaggcggggatgacgtcaaatcatcatgccccttatgagctgggctacacacgtgctacaatggatggtacaacgagcagcgatcccgcaagggctagctaatctttcaaagccattctcagttcggattgcactctgcaactcgagtgcatgaagtcggaatcgctagtaatcgcgaatcagaatgtcgcggtgaatccgttcccgggtcttgtacacaccgcccgtcacaccacgagagtttgaaacacccgaagtcggccggccaacccta

(19) United States
(12) Patent Application Publication
(10) Pub. No.: US 2025/0241966 A1
(43) Pub. Date: Jul. 31, 2025

(54) AIRWAY MEDICAMENTS FOR CRS

(71) Applicant: Trench Therapeutics, Inc., Palo Alto, CA (US)

(72) Inventors: David S. Harburger, Waltham, MA (US); Prashant Girinath, Arlington, MA (US)

(21) Appl. No.: 19/184,998

(22) Filed: Apr. 21, 2025

Related U.S. Application Data
(63) Continuation of application No. PCT/US23/77128, filed on Oct. 18, 2023.
(60) Provisional application No. 63/380,447, filed on Oct. 21, 2022.

(51) Int. Cl. A61K 35/74; A61P 31/04

(52) U.S. Cl. CPC A61K 35/74; A61P 31/04

(57) ABSTRACT

Described herein are compositions, methods, kits and devices for the treatment and/or prevention upper respiratory tract conditions, including sinusitis. In particular, bacterial populations described herein are live, purified bacteria for the modulation, restoration and/or promotion of the microbiome in the upper respiratory tract of a subject, including the nasal cavity, to promote health. Such bacterial populations may include single or multiple strains for bacteria. The multiple strains of bacteria may be strains from the same or different species, including species of Corynebacterium and/or Dolosigranulum pigrum.

### CROSS-REFERENCE TO RELATED APPLICATIONS

This application is a continuation application of International Application No. PCT/US2023/077128, filed Oct. 18, 2023, which claims the benefit of U.S. Provisional Application No. 63/380,447, filed Oct. 21, 2022 the disclosure of which is incorporated herein by reference in its entirety.

### SEQUENCE LISTING

The instant application contains a Sequence Listing which has been submitted electronically in ST.26 xml format and is hereby incorporated by reference in its entirety. Said xml copy, created on Oct. 10, 2023, is named 200546-010701.xml and is 5,748 bytes in size.

### BACKGROUND

Chronic rhinosinusitis (CRS) is an inflammatory disease of the paranasal sinuses that occurs in 1% to 5% of the U.S. population. Host and environmental factors influence the integrity of the microbiome in subjects. Microbial imbalance can lead to inflammation (or vice versa), resulting in opportunistic pathogenic microorganism colonization and subsequent disease conditions. Probiotic bacteria provide a therapeutic opportunity for addressing microbiome imbalance and disease related conditions. Typically, probiotic bacteria in clinical development to date have focused on gut colonizing bacteria. Thus, there is a need for forms of intervention which promote an improved microbiome environment in the upper respiratory tract, in particular the nasal cavity, for the treatment and prevention of conditions in the nasal cavity, including CRS. as well as in systems linked to the upper respiratory tract by a shared mucosal network.

### BRIEF SUMMARY

Provided herein are compositions, including pharmaceutical compositions, methods, kits and devices for modification of microbiome in a subject. As described in more detail herein, such compositions provide for, among other things, defending the integrity of the nasal cavity microbiome, and beneficially imparting downstream effects on the upper respiratory tract (including the nasal cavity). Further provided herein are mixtures of live, purified bacterial strains affording various bactericidal mechanisms to pathogenic bacteria, and in some instances the live, purified bacteria are mutualistic in their relationship to each other.

Disclosed herein are methods for treatment of chronic rhinosinusitis (CRS). In some embodiments, a method comprises: administering to a subject a live, purified population of bacteria, wherein the live, purified population of bacteria comprises: a plurality of strains of Dolosigranulum pigrum; and wherein the live, purified population of bacteria is present in an amount sufficient for treatment of CRS. In some embodiments, the live, purified population of bacteria comprises strains of Dolosigranulum pigrum comprising different bactericidal mechanisms to pathogenic bacteria. In some embodiments, the pathogenic bacteria comprises S. aureus, Streptococcus pneumoniae, Pseudomonas aeruginosa, Burkholderia pseudomallei, Haemophilus influenzae, or Moraxella catarrhalis. In some embodiments, the CRS is with nasal polyps (CRSwNP). In some embodiments, the CRS is without nasal polyps (CRSsNP). In some embodiments, the live, purified population of bacteria is present in an amount sufficient for reduction in colonization of a pathogenic bacteria in a nasal cavity of the subject. In some embodiments, the live, purified population of bacteria is present in an amount sufficient for reduction in colonization of a pathogenic bacteria in lung of the subject. In some embodiments, the live, purified population of bacteria is present in an amount sufficient for improvement of sense of smell in the subject. In some embodiments, the plurality of strains of Dolosigranulum pigrum comprises 1, 2, 3, 4, 5 or more strains listed in Table 2. In some embodiments, the live, purified population of bacteria further comprises at least one strain of Corynebacterium. In some embodiments, the live, purified population of bacteria further comprises a plurality of strains of Corynebacterium. In some embodiments, the Corynebacterium comprises C. accolens, C. pseudodiphtheriticum, C. amycolatum, C. propinquum, C. glutamicum, or C. striatum. In some embodiments, the Corynebacterium comprises Corynebacterium pseudodiphtheriticum. In some embodiments, the Corynebacterium pseudodiphtheriticum comprises JCM 1320 and/or ATCC 10700. In some embodiments, the live, purified population of bacteria is administered intranasally. In some embodiments, the subject is an infant. In some embodiments, the subject is a child. In some embodiments, the subject is an adult. In some embodiments, the live, purified population of bacteria is present in a total amount of up to 10\hat{ }15 cfu. In some embodiments, the live, purified population of bacteria is present in a total amount of at least 10\hat{ }3 cfu, optionally 10\hat{ }3 to 10\hat{ }12 cfu. In some embodiments, the subject has Cystic Fibrosis. In some embodiments, the administering is subsequent to the subject receiving endoscopic sinus surgery. In some embodiments, the subject has a S. aureus infection. In some embodiments, the subject has a recurrent S. aureus infection. In some embodiments, the subject has an at least 12 week history of CRS, an endoscopic evidence of CRS, a polypoid tissue Grade 1 or less, or any combination thereof.

Also disclosed herein are methods for treatment of sinusitis. In some embodiments, a method comprises: administering to a subject a live, purified population of bacteria, wherein the live, purified population of bacteria comprises: a plurality of strains of Corynebacterium pseudodiphtheriticum; and wherein the live, purified population of bacteria is present in an amount sufficient for treatment of sinusitis. In some embodiments, the live, purified population of bacteria comprises strains of Corynebacterium pseudodiphtheriticum comprising different bactericidal mechanisms to pathogenic bacteria. In some embodiments, the pathogenic bacteria comprises S. aureus, Streptococcus pneumoniae, Pseudomonas aeruginosa, Burkholderia pseudomallei, Haemophilus influenzae, or Moraxella catarrhalis. In some embodiments, the live, purified population of bacteria is present in an amount sufficient for reduction in colonization of a pathogenic bacteria in a nasal cavity of the subject. In some embodiments, the live, purified population of bacteria is present in an amount sufficient for reduction in colonization of a pathogenic bacteria in lung of the subject. In some embodiments, the live, purified population of bacteria is present in an amount sufficient for improvement of sense of smell in the subject. In some embodiments, the Corynebacterium pseudodiphtheriticum comprises JCM 1320 and/or ATCC 10700. In some embodiments, the live, purified population of bacteria is administered intranasally. In some embodiments, the subject is an infant. In some embodiments, the subject is a child. In some embodiments, the subject is an adult. In some embodiments, the live, purified population of bacteria is present in a total amount of up to 10\hat{ }15 cfu. In some embodiments, the live, purified population of bacteria is present in a total amount of at least 10\hat{ }3 cfu, optionally 10\hat{ }3 to 10\hat{ }12 cfu. In some embodiments, the subject has Cystic Fibrosis. In some embodiments. the administering is subsequent to the subject receiving endoscopic sinus surgery.

Also disclosed herein are methods for treatment of chronic rhinosinusitis (CRS). In some embodiments, the method comprises: administering to a subject a first therapy comprising an antibiotic, a steroid, or both; and administering to the subject a second therapy comprising a live, purified population of bacteria, wherein the live, purified population of bacteria comprises: a plurality of strains of Dolosigranulum pigrum. In some embodiments, the live, purified population of bacteria is present in an amount sufficient for treatment of CRS. In some embodiments, the first therapy and the second therapy are administered concurrently, or consecutively. In some embodiments, the first therapy is administered before the second therapy. In some embodiments, the antibiotic comprises a macrolide. In some embodiments, the macrolide comprises an azithromycin, a clarithromycin, an erythromycin, or a salt of any of these. In some embodiments, the steroid comprises a prednisone, a beclomethasone dipropionate, a triamcinolone acetonide, a flunisolide, a budesonide, a fluticasone propionate, a mometasone furoate, a ciclesonide, or a fluticasone furoate. In some embodiments, the first therapy is the antibiotic. In some embodiments, the first therapy is the steroid. In some embodiments, the live, purified population of bacteria comprises a plurality of strains of Dolosigranulum pigrum comprising different bactericidal mechanisms to pathogenic bacteria. In some embodiments, the pathogenic bacteria comprises S. aureus, Streptococcus pneumoniae, Pseudomonas aeruginosa, Burkholderia pseudomallei, Haemophilus influenzae, or Moraxella catarrhalis. In some embodiments, the CRS is with nasal polyps (CRSwNP). In some embodiments, the CRS is without nasal polyps (CRSsNP). In some embodiments, the live, purified population of bacteria is present in an amount sufficient for reduction in colonization of a pathogenic bacteria in a nasal cavity of the subject. In some embodiments, the live, purified population of bacteria is present in an amount sufficient for reduction in colonization of a pathogenic bacteria in a lung of the subject. In some embodiments, the live, purified population of bacteria is present in an amount sufficient for improvement of sense of smell in the subject. In some embodiments, the plurality of strains of Dolosigranulum pigrum comprises 1, 2, 3, 4, 5 or more strains listed in Table 2. In some embodiments, the live, purified population of bacteria further comprises at least one strain of Corynebacterium. In some embodiments, the live, purified population of bacteria further comprises a plurality of strains of Corynebacterium. In some embodiments, the Corynebacterium comprises C. accolens, C. pseudodiphtheriticum, C. amycolatum, C. propinquum, C. glutamicum, or C. striatum. In some embodiments, the Corynebacterium comprises Corynebacterium pseudodiphtheriticum. In some embodiments, the Corynebacterium pseudodiphtheriticum comprises JCM 1320 and/or ATCC 10700. In some embodiments, the live, purified population of bacteria is administered intranasally. In some embodiments, the subject is an infant. In some embodiments, the subject is a child. In some embodiments, the subject is an adult. In some embodiments, the live, purified population of bacteria is present in a total amount of up to 10\hat{ }15 cfu. In some embodiments, the live, purified population of bacteria is present in a total amount of at least 10\hat{ }3 cfu, optionally 10\hat{ }3 to 10\hat{ }12 cfu. In some embodiments, the subject has Cystic Fibrosis. In some embodiments, the administering is subsequent to the subject receiving endoscopic sinus surgery. In some embodiments, the subject has a S. aureus infection. In some embodiments, the subject has a recurrent S. aureus infection. In some embodiments, the subject has an at least 12 week history of CRS, an endoscopic evidence of CRS, a polypoid tissue Grade 1 or less, or any combination thereof.

### BRIEF DESCRIPTION OF THE DRAWINGS

FIG. 1: Illustrates, in some embodiments, various dosage forms described herein for oral 101 and/or intranasal 102 administration. Dosage forms illustrated include an inhaler 103 for aerosol administration, liquid 104 and capsule 105 for oral administration, and a spray bottle 106 for intranasal administration.

FIG. 2A, in some embodiments, illustrates a plot of OD600 measurements from growth of ATCC 10700, with OD600 for the Y axis and time in hours on the X axis.

FIG. 2B, in some embodiments, illustrates a plot of OD600 measurements from growth of ATCC 10700, with Log (OD600) for the Y axis and time in hours on the X axis.

FIG. 3A, in some embodiments, illustrates a plot of OD600 measurements from growth of JCM 1320, with OD600 for the Y axis and time in hours on the X axis.

FIG. 3B, in some embodiments, illustrates a plot of OD600 measurements from growth of JCM 1320, with Log (OD600) for the Y axis and time in hours on the X axis.

FIG. 4A, in some embodiments, illustrates a plot of OD600 measurements from growth of cultures having varying amounts of ATCC 10700 and JCM 1320, with OD600 for the Y axis and time in hours on the X axis.

FIG. 4B, in some embodiments, illustrates a plot of OD600 measurements from growth of cultures having varying amounts of ATCC 10700 and JCM 1320, with Log (OD600) for the Y axis and time in hours on the X axis.

FIG. 5, in some embodiments, is an image capture of an agar plate, showing four spottings of ATCC 10700 on the left, and four spottings of JCM 1320 on the right. The image capture was taken after 24 hours of growth.

FIG. 6, in some embodiments, is an image capture of an agar plate, showing columns of left to right with two spottings of ATCC 10700, two spottings of D. pigrum, two spottings of D. pigrum, and two spottings of JCM 1320. The image capture was taken after 24 hours of growth.

### DETAILED DESCRIPTION

Provided herein are composition, methods, kits and devices relating to upper respiratory tract colonizing bacteria for prevention and/or treatment of respiratory tract conditions and/or chronic rhinosinusitis. Furthermore, provided herein are (1) probiotic bacterial mixtures (2) excipients, dosage forms and routes of administration for such mixtures, (3) and conditions for treatment with such probiotic bacterial mixtures.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included, unless the context clearly dictates otherwise.

The terminology used herein is for the purpose of describing particular instances only and is not intended to be limiting of any embodiment. As used herein, the singular forms “a,” “an” and “the” are intended to include the plural forms as well, unless the context clearly indicates otherwise. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The term “subject” as used herein includes human and non-human mammals, including for example: a primate, cow, horse, pig, sheep, goat, dog, cat, or rodent, capable of being colonized by other organisms.

In some embodiments, provided herein are compositions which include bacteria having a percent identity based on 16S rRNA bacterial genetic sequence, a hypervariable region of the 16S rRNA, or whole genome comparison to a reference strain. Typically, comparison of the 16S rRNA bacterial genetic sequence allows a strain to be identified as within the same species as another strain by comparing sequences with known bacterial DNA sequences using NCBI BLAST search. The level of identity in relation to a nucleotide sequence may be determined for at least 20 contiguous nucleotides, for at least 30 contiguous nucleotides, for at least at least 40 contiguous nucleotides, for at least 50 contiguous nucleotides, for at least 60 contiguous nucleotides, or for at least 100 contiguous nucleotides. In some embodiments, the level of identity in relation to a nucleotide sequence is determined for the entire sequence searched. Percent identity may be at least 70%, 80%, 85%, 90%, 91%, 92%, 93%, 94%, 95%, 96%, 97%, 98%, 99%, or 100% to a reference bacterial 16S rRNA sequence, 16S rRNA V4 region sequence, or whole genome sequence. Percent identity may be at least 70%, 80%, 85%, 90%, 91%, 92%, 93%, 94%, 95%, 96%, 97%, 98%, 99%, or 100% to a reference bacteria 16S rRNA: V1 region, V2 region, V3 region, V5 region, V6 region, V7 region, V8 region or V9 region sequence.

Provided herein are compositions comprising live bacteria. Various techniques for determining if the bacteria is live include measuring membrane stability, transcription, translation, and cell division. In some embodiments, a live bacterium can comprise a bacterium that retains membrane stability. In some embodiments, a live bacterium can comprise a bacterium that is capable of transcription and translation. In some embodiments, a live bacterium can comprise a bacterium that is capable of cell division. In some embodiments, live bacteria can be determined by a culture dependent or a culture independent technique. In some cases, live bacteria can comprise an individual or a group of bacteria that can produce a colony-forming unit (cfu) when plated on stable growth media. In some embodiments, live and/or dead bacteria can be determined by imaging, for example with a live/dead stain. In some embodiments, a viability PCR based method can be used to determine live bacteria. In some cases, a metabolomic assay can be used to determine live bacteria.

In some embodiments, reference to a population of bacteria or a purified population refers to a plurality of bacteria. In some embodiments, a purified bacteria is enriched from a source sample. Compositions described herein can comprise about: 10%, 20%, 30%, 40%, 50%, 60%, 70% or more of a single strain of bacteria.

As used herein, a substance is “pure” or “substantially pure” if it is substantially free of other components. The terms “purify.” “purifying” and “purified”, when applied to a bacterium, can refer to a bacterium that has been separated from at least some of the components with which it was associated either when initially produced or generated (e.g., whether in nature or in an experimental setting), or during any time after its initial production. A bacterium or a bacterial population may be considered purified if it is isolated at or after production, such as from a material or environment containing the bacterium or bacterial population, or by passage through culture, and a purified bacterium or bacterial population may contain other materials up to at least about 10%, about 20%, about 30%, about 40%, about 50%, about 60%, about 70%, about 80%, about 90%, or above about 90% and still be considered “isolated.” Purified bacteria and bacterial populations can be more than at least about 80%, about 85%, about 90%, about 91%, about 92%, about 93%, about 94%, about 95%, about 96%, about 97%, about 98%, about 99%, or more than at least about 99% pure by weight (w/w). In the instance of microbial compositions provided herein, the one or more bacterial types (species or strains) present in the composition can be independently purified from one or more other bacteria produced and/or present in the material or environment containing the bacterial type. Microbial compositions and the bacterial components thereof are generally purified from residual habitat products.

An isolated bacterium may have been (1) separated from at least some of the components with which it was associated when initially obtained (whether in nature or in an experimental setting), and/or (2) produced, prepared, purified, and/or manufactured by the hand of man, e.g, using artificial culture conditions such as (but not limited to) culturing on a plate and/or in a fermenter. Isolated bacteria can include those bacteria that are cultured, even if such cultures are not monocultures. Isolated bacteria can be separated from at least about 10%, about 20%, about 30%, about 40%, about 50%, about 60%, about 70%, about 80%, about 90%, or more of the other components with which they were initially associated. Isolated bacteria can be more than about 80%, about 85%, about 90%, about 91%, about 92%, about 93%, about 94%, about 95%, about 96%, about 97%, about 98%, about 99%, or more than about 99% pure. A bacterial population of a biological sample provided herein can comprise one or more bacteria, which may then be isolated from such sample. Isolated bacteria may be provided in a form that is not naturally occurring.

### 1) Probiotic Bacterial Mixtures

The nasal cavity of the upper respiratory tract is a nutrient-poor, high-salinity niche where bacteria compete for limited resources. A healthy nasal microbiome prevents pathogenic microorganisms from colonization, harmful products from such colonization, inflammation, and generation of “leaky” cell-cell junctions, all of which can result in subsequent disease conditions in other organs along a shared mucosal network, including lungs. The nasal cavity provides an opportunity for imparting beneficial microbiome change to prevent and/or treat many disease conditions.

Bacteria described herein are to be used to control, treat, reduce, eliminate, and/or prevent pathogenic colonization by an organism on a subject and/or reduce inflammation. In some embodiments, provided herein are compositions for reducing prolonged inflammation. Compositions described herein may be administered or designed for delivery to particular locations of the subject, in particular, the upper respiratory tract, including the anterior nares, nasal cavity, and/or nasopharynx. Bacterial strains described herein may be isolated from the upper respiratory tract regions including, without limitation, anterior nares, nasal cavity, and/or nasopharynx.

Compositions comprising bacteria described herein are used to treat chronic rhinosinusitis (CRS). In some cases, the CRS is with nasal polyps. In some cases, the CRS is without nasal polyps. In some cases, a subject with CRS has Cystic Fibrosis. In some cases, bacteria described herein are administered and reduce colonization of pathogenic bacteria in the nasal cavity of a subject. In some cases, bacteria described herein are administered and reduce colonization of pathogenic bacteria in the lung of a subject. In some cases, bacteria described herein are administered and reduce colonization of pathogenic bacteria in the sinuses of a subject. In some cases, after administration of the bacteria described herein, a subject has improvement in their sense of smell as compared to before the administration of the bacteria. In some cases, a sense of smell can be measured by dynamic olfactometry. In some instances, the administering of bacteria described herein is subsequent to the subject receiving endoscopic sinus surgery. In some cases, the bacteria described herein are one or more strains of Dolosigranulum pigrum, or one or more of strains of Corynebacterium pseudodiphtheriticum. In some cases, the bacteria described herein are one or more strains of Dolosigranulum pigrum, and one or more strains of Corynebacterium pseudodiphtheriticum. In some cases, the bacteria described herein are a plurality of strains of Dolosigranulum pigrum, or a plurality of Corynebacterium pseudodiphtheriticum. In some cases, the bacteria described herein are a plurality of Dolosigranulum pigrum, and a plurality of strains of Corynebacterium pseudodiphtheriticum. In some cases, 2, 3, 4, 5, 6 or more different strains of bacteria each have different bactericidal mechanisms. Reference to different bactericidal mechanisms as used herein includes, for example, when two or more strains within a composition provide at least some overlapping bactericidal mechanisms in addition to different bactericidal mechanisms. Examples of different bactericidal mechanisms include, without limitation, iron sequestration, secretion systems (e.g., type IV secretion system, type VI secretion system, type VII secretion system), toxins that target cell wall synthesis, toxins that target ATP production, toxins that target DNA replication, small molecules, proteins, phages, nanotubes, and nutrient sequestration. Bacteria have a set of core genes that are shared between species of a genera of bacteria. In contrast to core genes, accessory genes are often variable across strains within a species. The accessory genome often encodes different bactericidal mechanisms.

In some embodiments, provided herein are compositions having bacterial populations having one or more species, and one or more strains for each of the one or more species. In some instances, a composition described herein includes 1, 2, 3, 4, 5, 6, 7, 8, 9, 10 or more species of bacteria. In some instances, a composition described herein includes 1, 2, 3, 4, 5, 6, 7, 8, 9, 10 or more strains of bacteria. Further provided herein are bacterial populations comprising at least one species of Corynebacterium. Further provided herein are bacterial populations comprising a plurality of species of Corynebacterium. Corynebacterium are gram-stain-positive bacteria, non-spore forming and nonmotile. Exemplary Corynebacterium species for inclusion in compositions described herein include: C. accolens, C. afermentans, C. ammoniagenes, C. amycolatum, C. argentoratense, C. aquaticum, C. auris, C, bovis, C, diphtheria, C. equi (now Rhodococcus equi), C. efficiens, C. flavescens, C. glucuronolyticum, C. glutamicum, C. granulosum, C. haemolyticum, C. halofytica, C. kroppenstedtii, C. jeikeium, C. macginleyi, C. matruchotii, C. minutissimum, C. parvum (Propionibacterium acnes), C. paurometabolum, C. propinquum, C. pseudodiphtheriticum (C. hofmannii), C. pseudotuberculosis, C. ovis, C. pyogenes—Trueperella pyogenes, C. urealyticum, C. renale, C. spec, C. striatum, C. tenuis, C. ulcerans, C. urealyticum, and C. xerosis. In some instances, a composition described herein comprises 1, 2, 3, 4, 5, 6, 7, 8, 9, 10 of the Corynebacterium strains listed in Table 1. In some embodiments, a population of bacteria described herein comprises a Corynebacterium strain having a 16S rRNA sequence of at least 90, 91, 92, 93, 94, 95, 96, 97, 98, 99, or 100% identity to that of a strain listed in Table 1. In some embodiments, a population of bacteria described herein comprises a Corynebacterium strain having a 16S rRNA sequence of at least 97% sequence identity with that of a strain listed in Table 1. The sequence identity may be based on a 16s rRNA sequence, 16s rRNA hypervariable region sequence, such as V4, or whole genome comparison. The population of bacteria may be part of a pharmaceutical composition. The bacteria may be live and purified. In some embodiments, a composition herein comprises a plurality of strains of C. accolens. In some cases, a plurality of strains of C. accolens comprises ATCC 49726, KPL1818, or a combination of any of these. In some embodiments, a composition herein comprises a plurality of strains of C. amycolatum. In some cases, a plurality of strains of C. amycolatum comprises HM-109, DSM1567, DSM6922, or a combination of any of these. Further provided herein are mixtures of live, purified Corynebacterium bacterial strains affording various bactericidal mechanisms to pathogenic bacteria.

TABLE 1
Corynebacterium strains

| Species | Strain name | Reference or Deposit |
|---|---|---|
| C. pseudodiphtheriticum | KPL1989 | GenBank: AXLR01000000 |
| C. pseudodiphtheriticum | DSM44287 | GenBank: GCA_000688415.1 |
| | | ATCC 10700 |
| C. pseudodiphtheriticum | 090104 | GenBank: AVFF01000000 |
| | | JCM 1320 |
| C. accolens | KPL1818 | GenBank: AXMA01000001.1 |
| C. accolens | CNCTC Th | ATCC 49726 |
| | 4/63 | |
| C. amycolatum | DSM6922 | ATCC 49368 |
| C. amycolatum | DSM1567 | MN175937 |
| C. amycolatum | SK46 | HM-109 |
| C. propinquum | DSM44285 | GCA_000375525.1 |
| C. glutamicum | DSM20300 | ATCC 13032 |
| C. striatum | DSM20668 | ATCC 6940 |

In some embodiments, provided herein are bacterial populations comprising at least one species of Dolosigranulum. Further provided herein are bacterial populations comprising a plurality of species of Dolosigranulum. In some embodiments, provided herein are populations of bacteria comprising at least one strain of Dolosigranulum pigrum. In some instances, a composition described herein comprises 1, 2, 3, 4, 5, 6, 7, 8, 9, 10 of the D. pigrum strains listed in Table 2. In some embodiments, a population of bacteria described herein comprises a D. pigrum strain having a 16S rRNA sequence of at least 90, 91, 92, 93, 94, 95, 96, 97, 98, 99, or 100% sequence identity to that of a strain listed in Table 2. In some embodiments, a population of bacteria described herein comprises a D. pigrum strain having a 16S rRNA sequence of at least 97% sequence identity with that of a strain listed in Table 2. The sequence identity may be based on a 16s rRNA sequence, 16s rRNA hypervariable region sequence, such as V4, or whole genome comparison. The population of bacteria may be part of a pharmaceutical composition. The bacteria may be live and purified. Further provided herein are mixtures of live, purified Dolosigranulum bacterial strains affording various bactericidal mechanisms to pathogenic bacteria.

TABLE 2
Dolosigranulum strains

| Species | Strain name | 16s rRNA sequence |
|---|---|---|
| D. pigrum | KPL1914 | |
| D. pigrum | CDC 39-95 | |
| D. pigrum | CDC 4294-98 | |
| D. pigrum | CDC 4420-98 | |
| D. pigrum | CDC 4545-98 | |
| D. pigrum | CDC 4709-98 | |
| D. pigrum | CDC 4199-99 | |
| D. pigrum | CDC 4791-99 | |
| D. pigrum | CDC 4792-99 | |
| D. pigrum | AMBR11\| | ATGAGAGTTTGATCTTGGCTCAGGACGAACGCTGGC |
| | LMG P-31124 | GGCATGCCTAATACATGCAAGTCGAACGATGATATC |
| | | ACTGCTTGCAGTGATTGATTAGTGGCGAACGGGTGA |
| | | GTAACACGTGAGGAACTTGCCCATGAGCGGGGGAC |
| | | AACATTCGGAAACGGATGCTAATACCCCATAGGTGG |
| | | ATTGGTCGCATGACGAATTCATTAAAGGTGGCTTTG |
| | | CTACCACTCATGGATAGCCTCGCGGCGTATTAGCTA |
| | | GTTGGTAAGGTAATGGCTTACCAAGGCAGTGATACG |
| | | TAGCCGACTTGAGAGGGTGATCGGCCACACTGGGAC |
| | | TGAGACACGGCCCAGACTCCTACGGGAGGCAGCAG |
| | | TAGGGAATCTTCCACAATGGGTGCAAACCTGATGGA |
| | | GCAATGCCGCGTGAGTGAAGAAGGTCTTCGGATCGT |
| | | AAAGCTCTGTTGTTAGAGAAGAACACGTGCTAGGTA |
| | | ACTACTAGCGCCTTGACGGTATCTAACCAGAAAGTC |
| | | ACGGCTAACTACGTGCCAGCAGCCGCGGTAATACGT |
| | | AGGTGACAAGCGTTGTCCGGATTTATTGGGCGTAAA |
| | | GGGAGCGCAGGCGGTCTGTTTAGTCTAATGTGAAAG |
| | | CCCACGGCTTAACCGTGGAACGGCATTGGAAACTGA |
| | | CAGACTTGAATGTAGAAGAGGAAAATGGAATTCCA |
| | | AGTGTAGCGGTGGAATGCGTAGATATTTGGAGGAAC |
| | | ACCAGTGGCGAAGGCGATTTTCTGGTCTAACATTGA |
| | | CGCTGAGGCTCGAAAGCGTGGGGAGCGAACAGGAT |
| | | TAGATACCCTGGTAGTCCACGCCGTAAACGATGAGT |
| | | GCTAAGTGTTGGGGGCTTTCCGGCCCTCGGTGCTGG |
| | | AGCTAACGTATTAAGCACTCCGCCTGGGGATTACGA |
| | | CCGCAAGGTTGAAACTCAAAAGAATTGACGGGGAC |
| | | CCGCACAAGCGGTGGAGCATGTGGTTTAATTCGAAG |
| | | CAACGCGAAGAACCTTACCAGCTCTTGACATCTTCT |
| | | GACAACTCTAGAGATAGAGCGTTCCCTTCGGGGACA |
| | | GAATGACAGGTGGTGCATGGTTGTCGTCAGCTCGTG |
| | | TCGTGAGATGTTGGGTTAAGTCCCGTAACGAGCGCA |
| | | ACCCTTGTTGTTAGTTGCCAGCATTAAGTTGGGCACT |
| | | TTAACGAGACTGCCGGTGATAAACCGGAGGAAGGC |
| | | GGGGATGACGTCAAATCATCATGCCCCTTATGAGCT |
| | | GGGCTACACACGTGCTACAATGGATGGTACAACGAG |
| | | CAGCGATCCCGCAAGGGCTAGCTAATCTTTCAAAGC |
| | | CATTCTCAGTTCGGATTGCACTCTGCAACTCGAGTGC |
| | | ATGAAGTCGGAATCGCTAGTAATCGCGAATCAGAAT |
| | | GTCGCGGTGAATCCGTTCCCGGGTCTTGTACACACC |
| | | GCCCGTCACACCACGAGAGTTTGAAACACCCGAAGT |
| | | CGGCCGGCCAACCCTATGGGAGGCAGCCGTCGAAG |
| | | GTGGGTTGAATGATTGGGGTGAAGTCGTAACAAGGT |
| | | AGCCGTATCGGAAGGTGCGGCTGGATCACCTCCTTT |
| | | (SEQ ID NO: 1) |
| D. pigrum | AMBR12\| | TCNCTGCTTGCAGTGANTGATTAGTGGCGAACGGGT |
| | LMG P-31154 | GAGTAACACGTGAGGAACTTGCCCATGAGCGGGGG |
| | | ACAACATTCGGAAACGGATGCTAATACCCCATAGGT |
| | | GGATTGGTCGCATGACGAATTCATTAAAGGTGGCTT |
| | | TGCTACCACTCATGGATAGCCTCGCGGCGTATTAGC |
| | | TAGTTGGTAAGGTAATGGCTTACCAAGGCAGTGATA |
| | | CGTAGCCGACTTGAGAGGGTGATCGGCCACACTGGG |
| | | ACTGAGACACGGCCCAGACTCCTACGGGAGGCAGC |
| | | AGTAGGGAATCTTCCACAATGGGTGCAAACCTGATG |
| | | GAGCAATGCCGCGTGAGTGAAGAAGGTCTTCGGATC |
| | | GTAAAGCTCTGTTGTTAGAGAAGAACACGTGCTAGG |
| | | TAACTACTAGCGCCTTGACGGTATCTAACCAGAAAG |
| | | TCACGGCTAACTACGTGCCAGCAGCCGCGGTAATAC |
| | | GTAGGTGACAAGCGTTGTCCGGATTTATTGGGCGTA |
| | | AAGGGAGCGCAGGCGGTCTGTTTAGTCTAATGTGAA |
| | | AGCCCACGGCTTAACCGTGGAACGGCATTGGAAACT |
| | | GACAGACTTGAATGTAGAAGAGGAAAAATGGAATT |
| | | CCAAGTGTAGCGGTGGAATGCGTAGATATTTGGAGG |
| | | AACACCAGTGGCGAAGGCGATTTTCTGGTCTAACAT |
| | | TGACGCTGAGGCTCGAAAGCGTGGGGAGCGAACAG |
| | | GATTAGATACCCTGGTAGTCCACGCCGTAAACGATG |
| | | AGTGCTAAGTGTTGGGGGCTTTCCGGCCCTCGGTGC |
| | | TGGAGCTAACGTATTAAGCACTCCGCCTGGGGATTA |
| | | CGACCGCAAGGTTGAAACTCAAAAGAATTGACGGG |
| | | GACCCGCACAAGCGGTGGAGCATGTGGTTTAATTCG |
| | | AAGCAACGCGAAGAACCTTACCAGCTCTTGACATCT |
| | | TCTGACAACTCTAGAGATAGAGCGTTCCCTTCGGGG |
| | | ACAGAATGACAGGTGGTGCATGGTTGTCGTCAGCTC |
| | | GTGTCGTGAGATGTTGGGTTAAGTCCCGTAACGAGC |
| | | GCAACCCTTGTTGTTAGTTGCCAGCATTAAGTTGGG |
| | | CACTTTAACGAGACTGCCGGTGATAAACCGGAGGAA |
| | | GGCGGGGATGACGTCAAATCATCATGCCCCTTATGA |
| | | GCTGGGCTACACACGTGCTACAATGGATGGTACAAC |
| | | GAGCAGCGATCCCGCAAGGGCTAGCTAATCTTTCAA |
| | | AGCCATTCTCAGTTCGGATTGCACTCTGCAACTCGA |
| | | GTGCATGAAGTCGGAATCGCTAGTAATCGCGAATCA |
| | | GAATGTCGCGGTGAATCCGTTCCCGGGTCTTGTACA |
| | | CACCGCCCGTCACACCACGAGAGTTTGAAACACCCG |
| | | AAGTCGGCCGGCCAACCCTA (SEQ ID NO: 2) |
| | | (an “N” nucleotide can be an A, T, G, or C) |

Further provided herein are populations of bacteria for colonization to the upper respiratory tract having a combination of strains including strains, from different species. In some embodiments, the populations of bacteria comprise at least one strain of Dolosigranulum, and optionally at least one strain of Corynebacterium. In some embodiments, the populations of bacteria comprise at least one strain of C. pseudodiphtheriticum and at least one strain of D. pigrum. In some embodiments, the populations of bacteria comprise at least one strain listed in Table 1, and at least one strain listed in Table 2. In some embodiments, the populations of bacteria comprise at least one strain having a 16S rRNA sequence of at least 97% sequence identity with that of a strain listed in Table 1, and at least one strain having a 16S rRNA sequence of at least 97% sequence identity with that of a strain listed in Table 2. In further embodiments, 1, 2, 3 or more of the Corynebacterium strains are C. pseudodiphtheriticum strains. The population of bacteria may be part of a pharmaceutical composition. The bacteria may be live and purified. Compositions described herein may have mixtures of species. The mixtures may be up to 1, 2, 3, 4, 5, 6, 7, 8, 9, 10 or more species and may include species listed in Table 1 and/or Table 2. Such species may be present in equal amounts or varied amounts. In some embodiments, each different species is present in at least 1%, 10%, 15%, 20%, 25%, 30%, 35%, 40%, 45%, 50%, or 100% of the total colony forming units (CFUs) for the total CFUs for the population of bacteria. Compositions described herein may have mixtures of strains within a species. The mixtures may be up to 1, 2, 3, 4, 5, 6, 7, 8, 9, 10 or more strains and may be from a species listed in Table 1 and/or Table 2. Such strains may be present in equal amounts or varied amounts. In some embodiments, different strains are present in at least 10%, 15%, 20%, 25%, 30%, 35%, 40%, 45%, 50%, or 100% of the total colony forming units (CFUs) for the total CFUs for the population of bacteria.

In some embodiments, provided herein are bacterial populations comprising at least one species of Lactobacillus. Further provided herein are bacterial populations comprising a plurality of species of Lactobacillus. In some embodiments, a composition herein comprises one or more of the following species of Lactobacillus: L. casei, L. plantarum, L. gasseri, L. crispatus, L. acidophilus, L. jensenii, L. fermentum, L. rhamnosus, L. pentosus, or L. sakei. In some embodiments, a composition herein comprises one or more strains of the following species of Lactobacillus: L. casei, L. plantarum, L. gasseri, L. crispatus, L. acidophilus, L. jensenii, L. fermentum, L. rhamnosus, L. pentosus, L. plantarum, or L. sakei.

In some instances, a composition described herein comprises 1, 2, 3, 4, 5, 6, 7, 8, 9, 10 of the Lactobacillus strains listed in Table 3. In some embodiments, a population of bacteria described herein comprises a Lactobacillus strain having a 16S rRNA sequence of at least 90, 91, 92, 93, 94, 95, 96, 97, 98, 99, or 100% sequence identity to that of a strain listed in Table 3. In some embodiments, a population of bacteria described herein comprises a Lactobacillus strain having a 16S rRNA sequence of at least 97% sequence identity with that of a strain listed in Table 3. The sequence identity may be based on a 16s rRNA sequence, a 16s rRNA hypervariable region sequence, such as V4, or whole genome comparison. The population of bacteria may be part of a pharmaceutical composition. The bacteria may be live and purified. Compositions described herein may have mixtures of species. The mixtures may be up to 1, 2, 3, 4, 5, 6, 7, 8, 9, 10 or more species and may include species listed in Table 1, Table 2, Table 3, or any combination thereof. Such species may be present in equal amounts or varied amounts. In some embodiments, each different species is present in at least 1%, 10%, 15%, 20%, 25%, 30%, 35%, 40%, 45%, 50%, or 100% of the total colony forming units (CFUs) for the total CFUs for the population of bacteria. Compositions described herein may have mixtures of strains within a species. The mixtures may be up to 1, 2, 3, 4, 5, 6, 7, 8, 9, 10 or more strains and may be from a species listed in Table 1, Table 2, Table 3, or any combination thereof. Such strains may be present in equal amounts or varied amounts. In some embodiments, different strains are present in at least 10%, 15%, 20%, 25%, 30%, 35%, 40%, 45%, 50%, or 100% of the total colony forming units (CFUs) for the total CFUs for the population of bacteria. The bacteria may be live and purified. Further provided herein are mixtures of live, purified Lactobacillus bacterial strains affording various bactericidal mechanisms to pathogenic bacteria.

TABLE 3
Lactobacillus and Lactococcus strains

| Species | Strain name | Reference and/or Deposit |
|---|---|---|
| L. casei | AMBR2 | Belgian Co-ordinated |
| | | Collection of Micro-Organisms |
| | | Deposit LMG P-30039 |
| | | Lab of Applied Microbiology |
| | | and |
| | | Biotechnology (LAMB) |
| | | PRJEB21025 |
| L. sakei | AMBR8 | LAMB PRJEB32716 |
| L. rhamnosus | GG | ATCC 53103 |

In some embodiments, provided herein are bacterial populations comprising at least one species of Lactococcus. Further provided herein are bacterial populations comprising a plurality of species of Lactococcus. In some embodiments, a composition herein comprises Lactococcus lactis. In some instances, a composition described herein comprises 1, 2, 3, 4, 5, 6, 7, 8, 9, 10 Lactococcus lactis stains. In some embodiments, a population of bacteria described herein comprises a Lactococcus lactis strain W136. In some embodiments, provided herein are compositions comprising a plurality of strains of D. pigrum and/or Corynebacterium pseudodiphtheriticum and one or more strains of Lactococcus lactis.

Corynebacteria are Gram-positive, non-motile, facultative anaerobes, characterized as having the appearance of straight or slightly curved slender rods with tapered or clubbed ends. Corynebacterium pseudodiphtheriticum, previously designated as Corynebacterium hofmannii, is a nonlipophilic, nonfermentive, urease- and nitrate-positive Corynebacterium species, which is part of the oropharyngeal bacterial flora. Corynebacterium accolens are Gram positive rods, irregularly shaped (‘coryneforms”), arranged as single cells, in pairs, in V forms, in palisades, or in clusters. Corynebacterium amycolatum, are Gram positive rods, irregularly shaped (‘coryneforms”), they are arranged as single cells, in pairs, in V forms, in palisades, or in clusters. Dolosigranulum pigrum is gram-positive, coccus arranged in pairs, tetrads, and clusters. Lactobacillus is a genus of Gram-positive, aerotolerant anaerobes or microaerophilic, rod-shaped, non-spore-forming bacteria.

In some embodiments, when administered to a subject, bacterial populations described herein reduce or eliminate colonization in the respiratory tract and/or sinuses of pathogenic bacteria. In some embodiments, when administered to a subject, bacterial populations described herein reduce abundance in the respiratory tract and/or sinuses of pathogenic bacteria. In some cases, reduction of colonization and/or abundance can be measured by the amount of pathogenic bacteria present before administration of the bacterial populations described herein as compared to the amount of pathogenic bacteria present after the administration of the bacterial populations described herein. Exemplary respiratory tract pathogenic bacteria include, without limitation, Staphylococcus aureus, Streptococcus pneumoniae, Pseudomonas aeruginosa, and Burkholderia pseudomallei. Exemplary strains of such pathogenic bacteria are listed in Table 4. Such reduction of pathogenic bacteria may be in the upper respiratory tract, the sinuses, or the lower respiratory tract.

TABLE 4
Pathogenic bacteria

| Species | Strain name | Reference or Deposit |
|---|---|---|
| S. aureus (MRSA) | JE2 | BEI Resources |
| | | NR-46543 |
| S. aureus (MRSA) | LAC | ATCC BAA-1556 |
| S. aureus (MRSA) | Mu50 | ATCC 700699 |
| S. aureus (MSSA) | 96: 308/USA900 | ATCC BAA-1749 |
| S. pneumoniae | TIGR4 | ATCC BAA-33 |
| S. pneumoniae | M270-8 | ATCC BAA-1659 |
| S. pneumoniae | DBL5 | AF071810 |
| Pseudomonas aeruginosa | | ATCC 27853 |
| Burkholderia pseudomallei | MSHR520 | GenBank: |
| | | GCA_000583835.1 |
| Streptococcus pneumoniae | (Klein) Chester | ATCC BAA-255 |
| Haemophilus influenzae | Lehmann and | ATCC 19418 |
| | Neumann | |
| Moraxella catarrhalis | (Frosch and Kolle) | ATCC 23246 |
| | Bovre | |

### 2) Excipients, Dosage Forms and Routes of Administrations

To facilitate administration, pharmaceutical compositions described herein may include one or more pharmaceutically acceptable excipients. Example pharmaceutically acceptable excipients include, without limitation, diluents, adjuvants, excipients, water, oils (including petroleum, animal, vegetable or synthetic oils.). Further examples include saline, gum acacia, gelatin, starch paste, talc, keratin, colloidal silica, and urea. Such excipients may include binders such as ethyl cellulose, carboxymethylcellulose, microcrystalline cellulose, or gelatin; excipients such as starch, lactose or dextrins; disintegrating agents such as alginic acid, sodium alginate, Primogel, and cornstarch; lubricants such as magnesium stearate or Sterotex; glidants such as colloidal silicon dioxide; sweetening agents such as sucrose or saccharin, a flavoring agent such as peppermint, methyl salicylate or orange flavoring, or coloring agents. Further examples of excipients include polyethylene glycol, cyclodextrin, oils, or any other similar liquid carrier that may be formulated into a capsule. Still further examples of excipients include sterile diluents such as water, saline solution, physiological saline, Ringer's solution, isotonic sodium chloride, fixed oils such as synthetic mono or digylcerides, polyethylene glycols, glycerin, cyclodextrin, propylene glycol or other solvents: antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose, thickening agents, lubricating agents, and coloring agents. In some embodiments of the invention, the pharmaceutically acceptable carrier can comprise a growth medium that can support the growth and/or static existence of beneficial bacteria described herein in the context of the pharmaceutical composition prior to administration of the pharmaceutical composition to the subject.

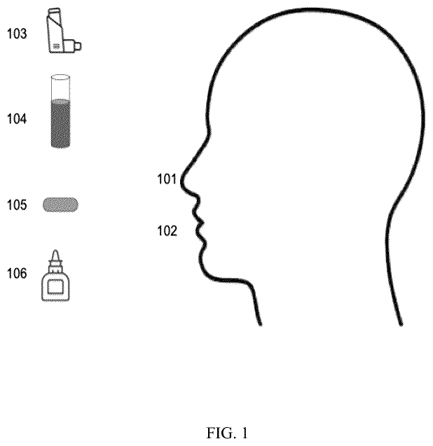

In some instances, a pharmaceutical composition described herein includes materials capable of modifying the physical form of a dosage unit. For example, various dosage forms described herein are illustrated in FIG. 1 for oral 101 and/or intranasal 102 administration. Dosage forms for compositions described herein include a nebulizer or inhaler 103 for aerosol administration, liquid 104 and capsule 105 for oral administration, and a spray bottle 106 for intranasal administration. In some instances, a pharmaceutical composition described herein is located within a nasal spray bottle. In some instances, a pharmaceutical composition described herein is prepared as an aerosol. Aerosols encompass a variety of systems including colloids and pressurized packages. Delivery of a composition in this form may include propulsion of a pharmaceutical composition including the beneficial bacteria described herein through use of liquefied gas or other compressed gas or by a suitable pump system. Aerosols may be delivered in single phase, bi-phasic, or triphasic systems. Compositions, including pharmaceutical compositions, described herein may be formulated depending on the route of administration. Such forms include, without limitation, solutions, suspensions, emulsions, cream, gel, lotion, ointment, tablets, tabs, films, pills, pellets, capsules, capsules including liquids, powders, sustained-release formulations, directed release formulations. Iyophylates (generated by freeze drying), emulsions, aerosols, sprays, granules, powders, or syrups. Dosage forms may be, without limitation, liquid, a solid, semisolid, gel, or aerosol. Methods of administration include, but are not limited to, oral, intranasal, or by inhalation. Pharmaceutical compositions described herein may include kits where bacteria described herein are included in a first container (e.g., lyophilized cells), and one or more pharmaceutical acceptable excipients are included in a second container (e.g., water).

Dosing may include single or multiple administrations of pharmaceutical compositions described herein. Examples include: multiple times a day, daily, every other day, 1, 2, 3, 5, 6, or 7 times a week, weekly, or less often, a single administration, a course of treatment involving several treatments on a regular or irregular basis, or multiple administrations for a period of time until a diminution of colonization is achieved. In some cases, dosing can occur every day, 2 days, 3 days, 4 days, 5 days, 6 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 1 year, 2 years, or as needed. The dosing regimen, including the regularity of and mode of administration, may be dependent on factors including but not limited to the subject being treated: the severity of the condition: the manner of administration, the stage of colonization, the presence of one or more other conditions such as pregnancy, infancy, or the presence of one or more additional diseases. In some embodiments, the subject is an infant. The infant can be up to 24 months old. In some embodiments, the subject is a child. The child may be 2 years to 21 years old. In some embodiments, the subject is an adult. Adults may be 21 years old or more. In some embodiments, the adult is of advanced age, such as 65 years or older.

Compositions, including pharmaceutical compositions, described herein may comprise a single (unit) dose of bacteria. Compositions described herein may comprise about 10² to about 10¹⁵ colony forming units (cfu) of bacteria or a bacterial strain described herein. Compositions described herein may comprise about: 10² to 10¹² cfu, 10³ to 10¹² cfu, 10³ to 10¹¹ cfu, 10³ to 10¹⁰ cfu, 10³ to 10⁹ cfu, 10³ to 10⁸ cfu, 10³ to 10⁷ cfu, 10³ to 10⁶ cfu, 10³ to about 10⁵ cfu, 10³ to 10⁴ cfu, 10⁴ to 10¹² cfu, 10⁴ to 10¹¹ cfu, 10⁴ to 10¹⁰ cfu, 10⁴ to 10⁹ cfu, 10⁴ to 10⁸ cfu, 10⁴ to 10⁷ cfu, 10⁴ to 10⁶ cfu, 10⁵ to 10¹² cfu, 10⁵ to 10¹¹ cfu, about 10⁵ to about 10¹⁰ cfu, 10⁶ to 10¹² cfu, 10⁷ to 10¹² cfu, 10⁸ to 10¹² cfu, 10⁹ to 10¹² cfu, 10¹⁰ to 10¹² cfu, 10¹¹ to 10¹² cfu, or 10⁶ to 10¹⁰ cfu of bacteria or a bacterial strain described herein. In some embodiments, compositions comprise about 10³ cfu, about 10⁴ cfu, about 10⁵ cfu, about 10⁶ cfu, about 10⁷ cfu, about 10⁸ cfu, about 10⁹ cfu, about 10¹⁰ cfu, about 10¹¹ cfu, or about 10¹² cfu of bacteria or a bacterial strain described herein.

Compositions, including pharmaceutical compositions, described herein may comprise 10² to 10¹⁵ colony forming units (cfu) of bacteria or a bacterial strain described herein per mL. Compositions described herein may comprise about 10² to 10¹² cfu, 10³ to 10¹² cfu, 10³ to 10¹¹ cfu, 10³ to 10¹⁰ cfu, 10³ to 10⁹ cfu, 10³ to 10⁸ cfu, 10³ to 10⁷ cfu, 10³ to 10⁶ cfu, 10³ to about 10⁵ cfu, 10³ to 10⁴ cfu, 10⁴ to 10¹² cfu, 10⁴ to 10¹¹ cfu, 10⁴ to 10¹⁰ cfu, 10⁴ to 10⁹ cfu, 10⁴ to 10⁸ cfu, 10⁴ to 10⁷ cfu, 10⁴ to 10⁶ cfu, 10⁵ to 10¹² cfu, 10⁵ to 10¹¹ cfu, about 10⁵ to about 10¹⁰ cfu, 10⁶ to 10¹² cfu, 10⁷ to 10¹² cfu, 10⁸ to 10¹² cfu, 10⁹ to 10¹² cfu, 10¹⁰ to 10¹² cfu, 10¹¹ to 10¹² cfu, or 10⁶ to 10¹⁰ cfu of bacteria or a bacterial strain described herein per mL.

Compositions described herein may comprise may at least about 0.01% by weight, at least about 0.05% by weight, at least about 0.1% by weight, at least about 0.2% by weight, at least about 0.3% by weight, at least about 0.4% by weight, at least about 0.5% by weight, at least about 0.6% by weight, at least about 0.7% by weight, at least about 0.8% by weight, at least about 0.9% by weight, at least about 1.0% by weight, at least about 1.5% by weight, at least about 2.0% by weight, at least about 3.0% by weight, at least about 4.0% by weight, at least about 5.0% by weight, at least about 6.0% by weight, at least about 7.0% by weight, at least about 8.0% by weight, at least about 9.0% by weight, at least about 10^{.0}% by weight, at least about 11.0% by weight, at least about 12.0% by weight, at least about 13.0% by weight, at least about 14.0% by weight, at least about 15.0% by weight, at least about 16.0% by weight, at least about 17.0% by weight, at least about 18.0% by weight, at least about 19.0% by weight, at least about 20.0% by weight, at least about 25.0% by weight, at least about 30.0% by weight, at least about 35.0% by weight, at least about 40.0% by weight, at least about 45.0% by weight, or at least about 50.0% by weight of bacteria or bacterial strain described herein. In some embodiments, compositions can include from 0.01% to 30% by weight, from about 0.01% to 20% by weight, from 0.01% to 5% by weight, from 0.1% to 30% by weight, from 0.1% to 20% by weight, from 0.1% to about 15% by weight, from 0.1% to 10% by weight, from 0.1% to 5% by weight, from 0.2% to 5% by weight, from 0.3% to 5% by weight, from 0.4% to 5% by weight, from 0.5% to 5% by weight, or from 1% to 5% by weight of bacteria or bacterial strain described herein.

Compositions, including pharmaceutical compositions, described herein may comprise a ratio (cfu to cfu) of about: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1:500, 1:600, 1:700, 1:800, 1:900 or about 1:1000 of a strain in Table 1 to another strain in Table 1 or a strain in Table 2 to another strain in Table 2. Compositions, including pharmaceutical compositions, described herein may comprise a ratio (cfu to cfu) of about: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1:500, 1:600, 1:700, 1:800, 1:900 or about 1:1000 of a strain in Table 1 to a strain in Table 2. Compositions, including pharmaceutical compositions, described herein may comprise a ratio (cfu to cfu) of about: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1:500, 1:600, 1:700, 1:800, 1:900 or about 1:1000 of multiple strains of Corynebacterium and/or Dolosigranulum pigrum.

### 3) Conditions

In some embodiments, provided herein are compositions and methods for the prevention or treatment of sinusitis. In some embodiments, provided herein are composition for use in the treatment of diseases, such as sinusitis and Chronic Rhinosinusitis (CRS). In some embodiments, provided herein are use of compositions in the manufacture of medicaments for the treatment of diseases, such as sinusitis and Chronic Rhinosinusitis (CRS). In some embodiments, the sinusitis is acute sinusitis (generally swelling of the sinuses for 4 weeks or less), chronic sinusitis (generally swelling of the sinuses for over 3 months), or subacute sinusitis (generally swelling of the sinuses for 1 to 3 months). In some embodiments, provided herein are compositions and methods for the prevention or treatment of Chronic Rhinosinusitis (CRS) or sinusitis, swelling or inflammation of the lining of the sinus. In some embodiments, the CRS is with nasal polyps (CRSwNP). In some embodiments, the CRS is without nasal polyps (CRSsNP). In some embodiments, the method provides for a reduction in occurrence of nasal polyps in the subject. In some embodiments, provided herein are methods for a reduction in occurrences or severity of pain or pressure in the forehead, nose, or between the eyes in a subject administered a composition described herein. In some embodiments, provided herein are methods for a reduction in occurrences or severity of headache in a subject administered a composition described herein. In some embodiments, provided herein are methods for a reduction in occurrences or severity of nasal discharge in a subject administered a composition described herein. In some embodiments, provided herein are methods for a reduction in occurrences or severity of coughing in a subject administered a composition described herein. In some embodiments, provided herein are methods for an improvement of olfactory function in a subject administered a composition described herein. In some embodiments, a composition described herein is administered to a subject prior to or after the subject has received endoscopic sinus surgery. In some embodiments, the subject has an at least 12 week history of CRS. In some cases, the subject has an at least 2, 3, 4, 5, 6, 7, 8, 9, 10, 11, 12, 13, 14, 15, 16, 17, 18, 19, or 20 week history of CRS. In some embodiments, the subject has endoscopic evidence of CRS. In some embodiments, the subject has polyploid tissue of Grade 1 or less. In some embodiments, the subject has polyploid tissue of Grade 2 or less. In some embodiments, the subject has polyploid tissue of Grade 3 or less. In some embodiments, the subject has polyploid tissue of Grade 4 or less. In some embodiments, the subject further has cystic fibrosis. In some embodiments, the subject further has a S. aureus infection. In some cases, the S. aureus infection is a recurrent S. aureus infection. In some embodiments, the composition provides for a reduction in inflammation in lung of the subject. In some embodiments, compositions described comprise beneficial bacteria present in an amount sufficient for a reduction in incidence of colonization of a pathogenic bacteria. In some embodiments, compositions described comprise beneficial bacteria present in an amount sufficient for a reduction in incidence of colonization of Streptococcus pneumoniae, Haemophilus influenzae, Moraxella catarrhalis or any combination thereof. In some embodiments, a pharmaceutical composition used to treat or prevent a respiratory condition described herein comprises at least one species (or strain) listed in Table 1 or Table 2, or a mixture listed in Tables 5-8. In some embodiments, a pharmaceutical composition used to treat or prevent a respiratory condition described herein comprises one or more strains of D. pigrum. In some embodiments, a pharmaceutical composition used to treat or prevent a respiratory condition described herein comprises one or more species of Corynebacterium. In some embodiments, a pharmaceutical composition used to treat or prevent a respiratory condition described herein comprises one or more strains of C. pseudodiphtheriticum. In some embodiments, a pharmaceutical composition used to treat or prevent a respiratory condition described herein comprises one or more strains of D. pigrum and one or more strains of C. pseudodiphtheriticum.

In some embodiments, an additional therapy can be administered to a subject, for example a steroid therapy, an antibiotic therapy or both. In some embodiments, a composition described herein is administered to a subject prior to or after the subject has received an antibiotic or a steroid. In some embodiments, a composition described herein is administered concurrently with an antibiotic or a steroid. In some cases, the antibiotic comprises a macrolide. In some cases, the macrolide comprises an azithromycin, a clarithromycin, an erythromycin. In some cases, the macrolide comprises an fidaxomicin, dirithromycin, a carbomycin, a josamycin, a kitasamycin, a midecamycin, a midecamycin acetate, an oleandomycin, a solithromycin, a spiramycin, a troleandomycin, or a roxithromycin. In some cases, the steroid comprises a prednisone, a beclomethasone dipropionate, a triamcinolone acetonide, a flunisolide, a budesonide, a fluticasone propionate, a mometasone furoate, a ciclesonide, or a fluticasone furoate.

### EXAMPLES

### Example 1: Bacterial Mixtures

i. Isolates and mixtures. Described herein are combinations of bacterial strains that can be used in generation of compositions, including pharmaceutical compositions for treatment of respiratory tract conditions. In the mixtures that follow, each of the live, purified strains are present in equal CFU amounts to other live, purified strains in each mixture.

First, mixtures having combinations of two strains are generated. Each mixture includes that two strains of Corynebacterium listed in Table 1, where an “X” denotes inclusion, are listed in the 44 mixtures (A1 to A44) in Table 5.

TABLE 5

| Mixture | KPL1989 | DSM44287 | 090104 | KPL1818 | DSM44278 | DSM6922 | DSM1567 | DSM44285 | DSM20300 | DSM20668 |
|---|---|---|---|---|---|---|---|---|---|---|
| A1 | X | X | | | | | | | | |
| A2 | X | | X | | | | | | | |
| A3 | X | | | X | | | | | | |
| A4 | X | | | | X | | | | | |
| A5 | X | | | | | X | | | | |
| A6 | X | | | | | | X | | | |
| A7 | X | | | | | | | X | | |
| A8 | X | | | | | | | | X | |
| A9 | X | | | | | | | | | X |
| A10 | | X | X | | | | | | | |
| A11 | | X | | X | | | | | | |
| A12 | | X | | | X | | | | | |
| A13 | | X | | | | X | | | | |
| A14 | | X | | | | | X | | | |
| A15 | | X | | | | | | X | | |
| A16 | | X | | | | | | | X | |
| A17 | | X | | | | | | | | X |
| A18 | | | X | X | | | | | | |
| A19 | | | X | | X | | | | | |
| A20 | | | X | | | X | | | | |
| A21 | | | X | | | | X | | | |
| A22 | | | X | | | | | X | | |
| A23 | | | X | | | | | | X | |
| A24 | | | X | | | | | | | X |
| A25 | | | | X | X | | | | | |
| A26 | | | | X | | X | | | | |
| A27 | | | | X | | | X | | | |
| A28 | | | | X | | | | X | | |
| A29 | | | | X | | | | | X | |
| A30 | | | | | X | X | | | | |
| A31 | | | | | X | | X | | | |
| A32 | | | | | X | | | X | | |
| A33 | | | | | X | | | | X | |
| A34 | | | | | X | | | | | X |
| A35 | | | | | | X | X | | | |
| A36 | | | | | | X | | X | | |
| A37 | | | | | | X | | | X | |
| A38 | | | | | | X | | | | X |
| A39 | | | | | | | X | X | | |
| A40 | | | | | | | | | X | |
| A41 | | | | | | | | | | X |
| A42 | | | | | | | | X | X | |
| A43 | | | | | | | | X | | X |
| A44 | | | | | | | | | X | X |

Second, mixtures are made that includes two strains of D. pigrum listed in Table 2. where an “X” denotes inclusion, are listed in the 66 mixtures (B1 to B66) in Table 6.

TABLE 6

| | | CDC39- | CDC2949- | CDC4294- | CDC4420- | CDC4545- | CDC4709- | CDC4199- | CDC4791- | CDC4791- | | |
|---|---|---|---|---|---|---|---|---|---|---|---|---|
| Mixture | KPL1914 | 95 | 98 | 98 | 98 | 98 | 98 | 99 | 99 | 99 | AMBR11 | AMBR12 |
| B1 | X | X | | | | | | | | | | |
| B2 | X | | X | | | | | | | | | |
| B3 | X | | | X | | | | | | | | |
| B4 | X | | | | X | | | | | | | |
| B5 | X | | | | | X | | | | | | |
| B6 | X | | | | | | X | | | | | |
| B7 | X | | | | | | | X | | | | |
| B8 | X | | | | | | | | X | | | |
| B9 | X | | | | | | | | | X | | |
| B10 | X | | | | | | | | | | X | |
| B11 | X | | | | | | | | | | | X |
| B12 | | X | X | | | | | | | | | |
| B13 | | X | | X | | | | | | | | |
| B14 | | X | | | X | | | | | | | |
| B15 | | X | | | | X | | | | | | |
| B16 | | X | | | | | X | | | | | |
| B17 | | X | | | | | | X | | | | |
| B18 | | X | | | | | | | X | | | |
| B19 | | X | | | | | | | | X | | |
| B20 | | X | | | | | | | | | X | |
| B21 | | X | | | | | | | | | | X |
| B22 | | | X | X | | | | | | | | |
| B23 | | | X | | X | | | | | | | |
| B24 | | | X | | | X | | | | | | |
| B25 | | | X | | | | X | | | | | |
| B26 | | | X | | | | | X | | | | |
| B27 | | | X | | | | | | X | | | |
| B28 | | | X | | | | | | | X | | |
| B29 | | | X | | | | | | | | X | |
| B30 | | | X | | | | | | | | | X |
| B31 | | | | X | X | | | | | | | |
| B32 | | | | X | | X | | | | | | |
| B33 | | | | X | | | X | | | | | |
| B34 | | | | X | | | | X | | | | |
| B35 | | | | X | | | | | X | | | |
| B36 | | | | X | | | | | | X | | |
| B37 | | | | X | | | | | | | X | |
| B38 | | | | X | | | | | | | | X |
| B39 | | | | | X | X | | | | | | |
| B40 | | | | | X | | X | | | | | |
| B41 | | | | | X | | | X | | | | |
| B42 | | | | | X | | | | X | | | |
| B43 | | | | | X | | | | | X | | |
| B44 | | | | | X | | | | | | X | |
| B45 | | | | | X | | | | | | | X |
| B46 | | | | | | X | X | | | | | |
| B47 | | | | | | X | | X | | | | |
| B48 | | | | | | X | | | X | | | |
| B49 | | | | | | X | | | | X | | |
| B50 | | | | | | X | | | | | X | |
| B51 | | | | | | X | | | | | | X |
| B52 | | | | | | | X | X | | | | |
| B53 | | | | | | | X | | X | | | |
| B54 | | | | | | | X | | | X | | |
| B55 | | | | | | | X | | | | X | |
| B56 | | | | | | | X | | | | | X |
| B57 | | | | | | | | X | X | | | |
| B58 | | | | | | | | X | | X | | |
| B59 | | | | | | | | X | | | X | |
| B60 | | | | | | | | X | | | | X |
| B61 | | | | | | | | | X | X | | |
| B62 | | | | | | | | | X | | X | |
| B63 | | | | | | | | | X | | | X |
| B64 | | | | | | | | | | X | X | |
| B65 | | | | | | | | | | X | | X |
| B66 | | | | | | | | | | | X | X |

Third, mixtures of Corynebacterium listed in Table 1 are each combined with one of the 12 strains of D. pigrum listed in Table 2, where an “X” denotes inclusion, are listed in the 66 mixtures (C1 to C130) in Table 7.

TABLE 7

| | Corynebacterium | | | | | | |
|---|---|---|---|---|---|---|---|
| | strain | | | | | | |
| Mixture | (below) | KPL1914 | CDC39-95 | CDC2949-98 | CDC4294-98 | CDC4420-98 | CDC4545-98 |
| C1 | KPL1989 | X | | | | | |
| C2 | DSM44287 | X | | | | | |
| C3 | 090104 | X | | | | | |
| C4 | KPL1818 | X | | | | | |
| C5 | DSM44278 | X | | | | | |
| C6 | DSM6922 | X | | | | | |
| C7 | DSM1567 | X | | | | | |
| C8 | DSM44285 | X | | | | | |
| C9 | DSM20300 | X | | | | | |
| C10 | DSM20668 | X | | | | | |
| C11 | KPL1989 | | X | | | | |
| C12 | DSM44287 | | X | | | | |
| C13 | 090104 | | X | | | | |
| C14 | KPL1818 | | X | | | | |
| C15 | DSM44278 | | X | | | | |
| C16 | DSM6922 | | X | | | | |
| C17 | DSM1567 | | X | | | | |
| C18 | DSM44285 | | X | | | | |
| C19 | DSM20300 | | X | | | | |
| C20 | DSM20668 | | X | | | | |
| C21 | KPL1989 | | | X | | | |
| C22 | DSM44287 | | | X | | | |
| C23 | 090104 | | | X | | | |
| C24 | KPL1818 | | | X | | | |
| C25 | DSM44278 | | | X | | | |
| C26 | DSM6922 | | | X | | | |
| C27 | DSM1567 | | | X | | | |
| C28 | DSM44285 | | | X | | | |
| C29 | DSM20300 | | | X | | | |
| C30 | DSM20668 | | | X | | | |
| C41 | KPL1989 | | | | X | | |
| C42 | DSM44287 | | | | X | | |
| C43 | 090104 | | | | X | | |
| C44 | KPL1818 | | | | X | | |
| C45 | DSM44278 | | | | X | | |
| C46 | DSM6922 | | | | X | | |
| C47 | DSM1567 | | | | X | | |
| C48 | DSM44285 | | | | X | | |
| C49 | DSM20300 | | | | X | | |
| C50 | DSM20668 | | | | X | | |
| C51 | KPL1989 | | | | | X | |
| C52 | DSM44287 | | | | | X | |
| C53 | 090104 | | | | | X | |
| C54 | KPL1818 | | | | | X | |
| C55 | DSM44278 | | | | | X | |
| C56 | DSM6922 | | | | | X | |
| C57 | DSM1567 | | | | | X | |
| C58 | DSM44285 | | | | | X | |
| C59 | DSM20300 | | | | | X | |
| C60 | DSM20668 | | | | | X | |
| C61 | KPL1989 | | | | | | X |
| C62 | DSM44287 | | | | | | X |
| C63 | 090104 | | | | | | X |
| C64 | KPL1818 | | | | | | X |
| C65 | DSM44278 | | | | | | X |
| C66 | DSM6922 | | | | | | X |
| C67 | DSM1567 | | | | | | X |
| C68 | DSM44285 | | | | | | X |
| C69 | DSM20300 | | | | | | X |
| C70 | DSM20668 | | | | | | X |
| C71 | KPL1989 | | | | | | |
| C72 | DSM44287 | | | | | | |
| C73 | 090104 | | | | | | |
| C74 | KPL1818 | | | | | | |
| C75 | DSM44278 | | | | | | |
| C76 | DSM6922 | | | | | | |
| C77 | DSM1567 | | | | | | |
| C78 | DSM44285 | | | | | | |
| C79 | DSM20300 | | | | | | |
| C80 | DSM20668 | | | | | | |
| C81 | KPL1989 | | | | | | |
| C82 | DSM44287 | | | | | | |
| C83 | 090104 | | | | | | |
| C84 | KPL1818 | | | | | | |
| C85 | DSM44278 | | | | | | |
| C86 | DSM6922 | | | | | | |
| C87 | DSM1567 | | | | | | |
| C88 | DSM44285 | | | | | | |
| C89 | DSM20300 | | | | | | |
| C90 | DSM20668 | | | | | | |
| C91 | KPL1989 | | | | | | |
| C92 | DSM44287 | | | | | | |
| C93 | 090104 | | | | | | |
| C94 | KPL1818 | | | | | | |
| C95 | DSM44278 | | | | | | |
| C96 | DSM6922 | | | | | | |
| C97 | DSM1567 | | | | | | |
| C98 | DSM44285 | | | | | | |
| C99 | DSM20300 | | | | | | |
| C100 | DSM20668 | | | | | | |
| C101 | KPL1989 | | | | | | |
| C102 | DSM44287 | | | | | | |
| C103 | 090104 | | | | | | |
| C104 | KPL1818 | | | | | | |
| C105 | DSM44278 | | | | | | |
| C106 | DSM6922 | | | | | | |
| C107 | DSM1567 | | | | | | |
| C108 | DSM44285 | | | | | | |
| C109 | DSM20300 | | | | | | |
| C110 | DSM20668 | | | | | | |
| C111 | KPL1989 | | | | | | |
| C112 | DSM44287 | | | | | | |
| C113 | 090104 | | | | | | |
| C114 | KPL1818 | | | | | | |
| C115 | DSM44278 | | | | | | |
| C116 | DSM6922 | | | | | | |
| C117 | DSM1567 | | | | | | |
| C118 | DSM44285 | | | | | | |
| C119 | DSM20300 | | | | | | |
| C120 | DSM20668 | | | | | | |
| C121 | KPL1989 | | | | | | |
| C122 | DSM44287 | | | | | | |
| C123 | 090104 | | | | | | |
| C124 | KPL1818 | | | | | | |
| C125 | DSM44278 | | | | | | |
| C126 | DSM6922 | | | | | | |
| C127 | DSM1567 | | | | | | |
| C128 | DSM44285 | | | | | | |
| C129 | DSM20300 | | | | | | |
| C130 | DSM20668 | | | | | | |

| Mixture | CDC4709-98 | CDC4199-99 | CDC4791-99 | CDC4791-99 | AMBR11 | AMBR12 |
|---|---|---|---|---|---|---|
| C1 | | | | | | |
| C2 | | | | | | |
| C3 | | | | | | |
| C4 | | | | | | |
| C5 | | | | | | |
| C6 | | | | | | |
| C7 | | | | | | |
| C8 | | | | | | |
| C9 | | | | | | |
| C10 | | | | | | |
| C11 | | | | | | |
| C12 | | | | | | |
| C13 | | | | | | |
| C14 | | | | | | |
| C15 | | | | | | |
| C16 | | | | | | |
| C17 | | | | | | |
| C18 | | | | | | |
| C19 | | | | | | |
| C20 | | | | | | |
| C21 | | | | | | |
| C22 | | | | | | |
| C23 | | | | | | |
| C24 | | | | | | |
| C25 | | | | | | |
| C26 | | | | | | |
| C27 | | | | | | |
| C28 | | | | | | |
| C29 | | | | | | |
| C30 | | | | | | |
| C41 | | | | | | |
| C42 | | | | | | |
| C43 | | | | | | |
| C44 | | | | | | |
| C45 | | | | | | |
| C46 | | | | | | |
| C47 | | | | | | |
| C48 | | | | | | |
| C49 | | | | | | |
| C50 | | | | | | |
| C51 | | | | | | |
| C52 | | | | | | |
| C53 | | | | | | |
| C54 | | | | | | |
| C55 | | | | | | |
| C56 | | | | | | |
| C57 | | | | | | |
| C58 | | | | | | |
| C59 | | | | | | |
| C60 | | | | | | |
| C61 | | | | | | |
| C62 | | | | | | |
| C63 | | | | | | |
| C64 | | | | | | |
| C65 | | | | | | |
| C66 | | | | | | |
| C67 | | | | | | |
| C68 | | | | | | |
| C69 | | | | | | |
| C70 | | | | | | |
| C71 | X | | | | | |
| C72 | X | | | | | |
| C73 | X | | | | | |
| C74 | X | | | | | |
| C75 | X | | | | | |
| C76 | X | | | | | |
| C77 | X | | | | | |
| C78 | X | | | | | |
| C79 | X | | | | | |
| C80 | X | | | | | |
| C81 | | X | | | | |
| C82 | | X | | | | |
| C83 | | X | | | | |
| C84 | | X | | | | |
| C85 | | X | | | | |
| C86 | | X | | | | |
| C87 | | X | | | | |
| C88 | | X | | | | |
| C89 | | X | | | | |
| C90 | | X | | | | |
| C91 | | | X | | | |
| C92 | | | X | | | |
| C93 | | | X | | | |
| C94 | | | X | | | |
| C95 | | | X | | | |
| C96 | | | X | | | |
| C97 | | | X | | | |
| C98 | | | X | | | |
| C99 | | | X | | | |
| C100 | | | X | | | |
| C101 | | | | X | | |
| C102 | | | | X | | |
| C103 | | | | X | | |
| C104 | | | | X | | |
| C105 | | | | X | | |
| C106 | | | | X | | |
| C107 | | | | X | | |
| C108 | | | | X | | |
| C109 | | | | X | | |
| C110 | | | | X | | |
| C111 | | | | | X | |
| C112 | | | | | X | |
| C113 | | | | | X | |
| C114 | | | | | X | |
| C115 | | | | | X | |
| C116 | | | | | X | |
| C117 | | | | | X | |
| C118 | | | | | X | |
| C119 | | | | | X | |
| C120 | | | | | X | |
| C121 | | | | | | X |
| C122 | | | | | | X |
| C123 | | | | | | X |
| C124 | | | | | | X |
| C125 | | | | | | X |
| C126 | | | | | | X |
| C127 | | | | | | X |
| C128 | | | | | | X |
| C129 | | | | | | X |
| C130 | | | | | | X |

Fourth, a mixture of multiple Corynebacterium strains from Table 1 is made. In this example, 2, 3, 4, or 5 strains are from difference species of Corynebacterium are in the composition. Strains are selected from: C. pseudodiphtheriticum ATCC 10700 and/or JCM 1320; C. amycolatum ATCC 49358; C. glutamicum ATCC 13032; and C. striatum ATCC 6940. The compositions may include ATCC 10700 and/or JCM 1320 in addition to the strains from species other than ATCC 10700 and/or JCM 1320.

Fifth, a mixture of two Corynebacterium pseudodiphtheriticum strains from Table 1 is made: ATCC 10700 and JCM 1320, “mixture D1.”

Sixth, mixture D1 is combined with one of the 12 strains of D. pigrum listed in Table 2, where an “X” denotes inclusion, are listed in the 66 mixtures (E1 to E12) in Table 8.

TABLE 8

| | Mixture D1 | | | | | | |
|---|---|---|---|---|---|---|---|
| | (ATCC | | | | | | |
| | 10700 | | | | | | |
| | and JCM | | | | | | |
| Mixture | 1320) | KPL1914 | CDC39-95 | CDC2949-98 | CDC4294-98 | CDC4420-98 | CDC4545-98 |
| E1 | X | X | | | | | |
| E2 | X | | X | | | | |
| E3 | X | | | X | | | |
| E4 | X | | | | X | | |
| E5 | X | | | | | X | |
| E6 | X | | | | | | X |
| E7 | X | | | | | | |
| E8 | X | | | | | | |
| E9 | X | | | | | | |
| E10 | X | | | | | | |
| E11 | X | | | | | | |
| E12 | X | | | | | | |

| Mixture | CDC4709-98 | CDC4199-99 | CDC4791-99 | CDC4791-99 | AMBR11 | AMBR12 |
|---|---|---|---|---|---|---|
| E1 | | | | | | |
| E2 | | | | | | |
| E3 | | | | | | |
| E4 | | | | | | |
| E5 | | | | | | |
| E6 | | | | | | |
| E7 | X | | | | | |
| E8 | | X | | | | |
| E9 | | | X | | | |
| E10 | | | | X | | |
| E11 | | | | | X | |
| E12 | | | | | | X |

Seventh mixtures of Corynebacterium listed in Table 1 are combined to include at least one strain of each of C. pseudodiphtheriticum, C. accolens, and C. amycolatum. For example: ATCC 10700 and/or JCM 1320 plus ATCC 49726 and ATCC 49368.

iii. Cultivation from frozen stocks. Bacterial strains are grown at 37° C. with 5% CO2. D. pigrum strains are cultivated from frozen stocks on BBL Columbia Colistin-Nalidixic Acid (CNA) agar with 5% sheep blood (BD Diagnostics) for 2 days. Corynebacterium species are cultivated from frozen stocks on BHI agar (e.g., for C. pseudodiphtheriticum and C. propinquum) or BHI agar supplemented with 1% Tween 80 (e.g., for C. accolens) for 1 day. Resuspensions described below are made by harvesting colonies from agar medium and resuspending in 1× phosphate buffered saline (PBS).

### Example 2: MRSA Model Systems

i. Contact-Dependent Assays. Clinical isolates of C. pseudodiphtheriticum and D. pigrum taken from subjects (e.g., from strains listed in Table 1 and Table 2, and mixtures from Example 1) are assayed to determine if anti-S. aureus activity is dependent on direct physical contact between the bacteria. Briefly, a sterile 0.2 μm filter disk is placed on top of the BHIT agar (Brain Heart Infusion (BHI) agar (Becton Dickinson)) seeded with one of the S. aureus strains provided in Table 4 (JE2, LAC, Mu50, or USA 900). Each clinical isolate in a suspension is individually spotted on top of the filter disk so that none of the cell suspension physically touched the S. aureus seeded agar plate. Plates are incubated at 28° C. and visually assessed at 24, 72, and 120 hours for the absence or presence of a zone of clearance (ZOC). The absence of a ZOC in the presence of a filter disk indicates that physical contact is necessary for anti-S. aureus activity against the corresponding most sensitive S. aureus strain.

ii. Conditioned Cell Free Medium (CCFM) Preparation and Disk Diffusion Assays. Clinical isolates that produce contact-independent bactericidal anti-S. aureus activity (e.g., from strains listed in Table 1, Table 2, and mixtures from Example 1) are independently cultured in 10 mL BHIT broth overnight at 37° C, with shaking at 190 rpm. Cultures are pelleted by centrifugation, and the supernatant is filter-sterilized with a 2 μm filter (Corning). One milliliter of sterile supernatant is retained, and the remaining supernatant is concentrated (50×) with ammonium sulfate precipitation. For heat-treatments, 50 μL aliquots of unconcentrated or 50× CCFM are incubated at 90° C, for 10 minutes, then allowed to cool. For the disk diffusion assays, the S. aureus strain that is most sensitive to the corresponding inhibitory activity is cultured on BHI agar overnight at 37° C. The following day, the plate-grown cells are recovered and diluted to 1×10\hat{ }8 cells/ml (OD600 of 0.1) in BHI broth. A sterile swab is then used to spread the S. aureus cell suspension on BHIT agar as a lawn. The plate is allowed to dry in a laminar flow hood for 30 minutes. Next, a sterile 5 mm diffusion disk is placed on top of the S. aureus lawn, and 50 μL of unconcentrated CCFM or 50× CCFM is inoculated onto the disk. Plates are incubated at 28° C., and images are taken after 72 hours of incubation.

iii. S. aureus Infection and CCFM Treatment of Galleria mellonella Caterpillars.

Staphylococcus aureus strains JE2, LAC, Mu50, or USA 900 are cultured overnight on BHI agar at 37° C. The following day, S. aureus cells are recovered and diluted to 1×10\hat{ }8 cells/ml (OD600=0.1) in PBS. Total CFU are then further adjusted to obtain the required doses; i.e., 10\hat{ }7 CFU or 10\hat{ }6 CFU in 5 μL of PBS+0.01% bromophenol dye. For infections, Galleria mellonella caterpillars (Vanderhorst Wholesale Inc) are utilized within 1 day of receipt. Caterpillars between 200 and 300 mg are chosen for infection. Briefly, 5 μL of inoculum that contained 10\hat{ }7 or 10\hat{ }6 total CFU of S. aureus are injected into the last left proleg using a 10 μL glass syringe (Hamilton) fitted with a 31G needle. For caterpillars that are treated with CCFM obtains from strains in Table 1, Table 2, and mixtures from Example 1, the caterpillars are maintained at room temperature for 1 hour following the S. aureus injection, then refrigerated at 4° C, for 12 minutes and then injected with 5 μL of freshly prepared 50× CCFM from the clinical isolate (treated) or 50× concentrated BHIT (sham treated). These injections are into the last right proleg. All caterpillars are incubated at 37° C., and survival was monitored over 120 hours. Untouched, and PBS injected caterpillars are included as controls.

iv. Intranasal colonization assay for Methicillin-Resistant Staphylococcus aureus in Mice. Staphylococcus aureus strains JE2, LAC, Mu50, or USA 900 are cultured at 37° C. in either Todd-Hewitt broth (THB) or on Todd-Hewitt agar (THA) (Difco). Brain Heart Infusion (BHI) (Difco) broth and agar are used to grow C. pseudodiphtheriticum and D. pigrum strains (e.g., from strains listed in Table 1, Table 2, and Example 1). CD1 mice (Charles River Laboratories, Wilmington, MA) are obtained. Mice are inoculated intranasally with 10 μl droplet of the inocula at the indicated concentrations. Mice are administered 1×10\hat{ }9 CFU total of the bacteria. CD1 mice are inoculated intranasally with: (i) C. pseudodiphtheriticum, (ii) D. pigrum, (iii) C. pseudodiphtheriticum and D. pigrum, or (iv) PBS. After two days, the mice are administered streptomycin-resistant MRSA (JE2, LAC, Mu50, or USA 900) by the intranasal route, and sacrificed after another 2 days. For bacterial enumeration, the mice are euthanized using isoflurane followed by cervical dislocation, and the nasal tissue is homogenized and vortexed for 5 min in PBS, and the homogenate is plated on THA with or without streptomycin after appropriate serial dilutions. Bacterial identification is based on antibiotic resistance patterns, colony morphology, and color.

### Example 3: Pneumonia Model Systems

i. Growth assay of S. pneumoniae in cell-free conditioned liquid medium. After growth in BHI, D. pigrum or C. pseudodiphtheriticum cells (e.g., from strains listed in Table 1, Table 2, and mixtures from Example 1) are removed with a 0.22-μM sterile filter yielding cell-free conditioned medium (CFCM). The pH of the CFCM is adjusted using 2N H2SO4 and 10M KOH to match that of BHI broth alone within 0.02 pH units. S. pneumoniae strains TIGR4, DBL5, and M270-8 (see Table 4) are each grown on BBL Columbia CNA agar with 5% sheep blood for 1 day, harvested with a sterile cotton swab, resuspended to an OD600 of 0.30 in 1× PBS, inoculated at 1:100 into both of D. pigrum or C. pseudodiphtheriticum CFCM and BHI broth and grown for 19-20 hour at 37° C. in static (S. pneumoniae) culture under atmospheric conditions. Growth yield is quantified as OD600 absorbance.

ii. Growth assay for S. pneumoniae in conditioned by mono- vs. co-culture medium. D. pigrum and C. pseudodiphtheriticum strains (e.g., from strains listed in Table 1, Table 2, and mixtures from Example 1) are grown from freezer stocks. Cells are harvested with sterile cotton swabs and resuspended in sterile PBS to an OD600 nm of 0.5. Cells are then spotted in 100 μl of 1:1 mixed resuspension on a polycarbonate membrane on BHI agar medium containing 400 U/mL bovine liver catalase. After 2 days of growth, the polycarbonate membrane with D. pigrum and/or C. pseudodiphtheriticum is removed from each plate leaving cell-free conditioned agar medium. S. pneumoniae is grown overnight on BBL Columbia CNA agar with 5% sheep blood using a sterile cotton swab, a lawn is streaked onto the cell-free conditioned agar medium and allowed to grow for 24 hours. Growth/inhibition is assessed daily and photographically recorded.

iii. Mouse model. 6- to 8-week-old FVB/N mice are orally gavaged with 200 μL of either (i) C. pseudodiphtheriticum, (ii) D. pigrum, (iii) C. pseudodiphtheriticum and D. pigrum (e.g., from strains listed in Table 1, Table 2, and mixtures from Example 1), or (iv) sterile water (vehicle) (for the bacteria: 1×10\hat{ }9) colony-forming units (CFUs)/mL) immediately before procedure. Pneumonia is induced via direct intratracheal instillation of Pseudomonas aeruginosa (ATCC 27853) or S. pneumoniae (TIGR4, M270-8, or DBL5). Under isoflurane anesthesia, mice receive a midline cervical incision, and P. aeruginosa or S. pneumoniae is introduced into the trachea via a 29-gauge syringe. Forty microliters of 4×10\hat{ }8 CFUs of bacteria diluted in sterile saline is used. Mice are then held vertically for 5 seconds to enhance the delivery into the lungs. Sham mice are treated identically except that they receive an intratracheal injection of saline. All mice receive antibiotic therapy (gentamicin 0.2 mg/mL, subcutaneously) after the surgery to mimic clinical setting. Animals are killed at either 12 or 24 hours (for acute studies) or followed 7 days for survival. For acute studies, mice receive a single dose of (i) C. pseudodiphtheriticum, (ii) D. pigrum, or (iii) C. pseudodiphtheriticum and D. pigrum. For survival studies, mice are treated with (i) C. pseudodiphtheriticum, (ii) D. pigrum, or (iii) C. pseudodiphtheriticum and D. pigrum daily for 7 days and receive antibiotic treatment at 0, 12, and 24 hours. Lungs are tested for the presence of P. aeruginosa or S. pneumoniae.

### Example 4: Rhinitis Model System

Female BALB/cA mice 2 months old are used. Animals are fed a standard rodent chow diet in a temperature-controlled room (23 degrees C.) on a 12 hour light/dark cycle. The immune response of the mice is suppressed by subcutaneous injections of hydrocortisone (Hydrocortisone hemisuccinate 100, Polfa, PL, 100 mg/kg/day) at day 0 and day 4. S. aureus (JE2, LAC, Mu50, or USA 900) is applied intranasally on day 5. Bacterial treatment is performed on day 10. Forty microliters of 4×10\hat{ }8 CFUs of bacteria diluted in sterile saline is used. Mice receive a single dose of (i) C. pseudodiphtheriticum, (ii) D. pigrum, or (iii) C. pseudodiphtheriticum and D. pigrum (e.g., from strains listed in Table 1, Table 2, and mixtures from Example 1); or (iv) saline (vehicle). Animals are killed at either 12 or 24 hours or followed 7 days for survival, and tissue samples are taken. Blood and internal organ (e.g., lungs, kidney, liver and spleen) samples are tested for the presence of S. aureus; nasal epithelium is tested for carriage of S. aureus in control and experimental animals.

### Example 5: Corynebacterium Growth Assay

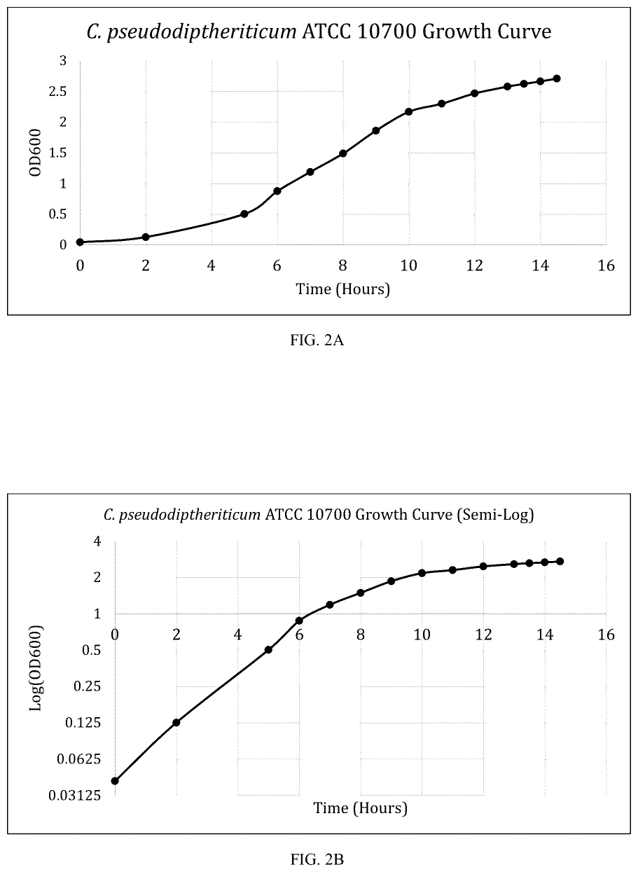

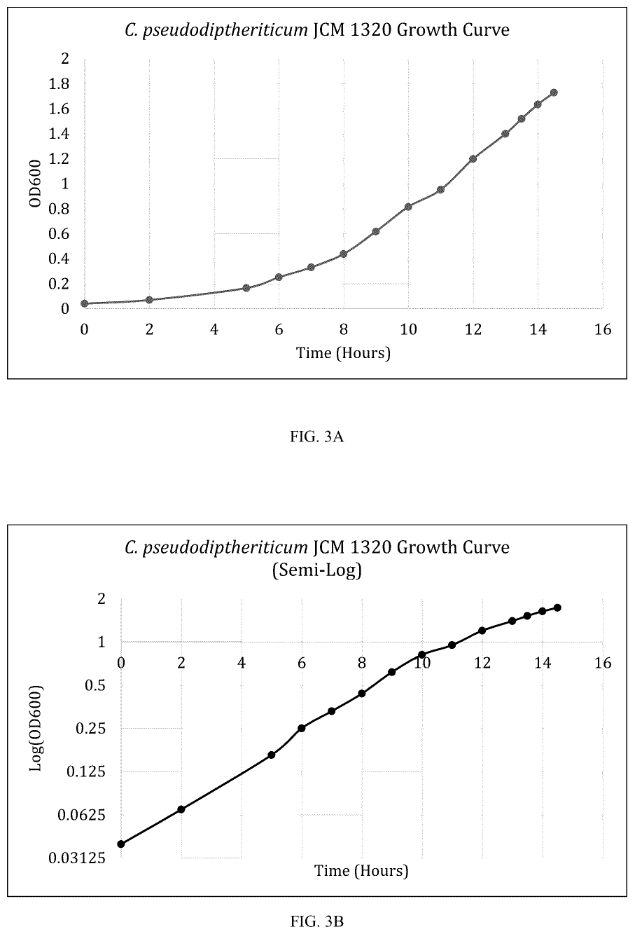

The following was performed to assay growth rate attributes for C. pseudodiptheriticum strains. 9 mL of Columbia 1% Tween broth was inoculated with C. pseudodiptheriticum colonies and incubated overnight at 37 degrees Celsius, shaking at 200 rpm. From the overnight culture, an OD600 measurement was taken and 30 mL of fresh Columbia 1% Tween broth was inoculated to reach a starting OD600 of 0.04, cultures were incubated at 37 degrees Celsius shaking at 200 rpm. OD600 measurements were taken over a 14.5 hour time period. Results of OD600 measurements for growth of ATCC 10700 are show in in FIG. 2A, and shown as Log (OD600) for the Y axis in FIG. 2B. As can be seen, the doubling time during log phase (hours 2 to 6) was about 86 minutes. Results of OD600 measurements for growth of JCM 1320 are show in in FIG. 3A, and shown as Log (OD600) for the Y axis in FIG. 3B. As can be seen, the doubling time during log phase (hours 2 to 6) was about 129 minutes.

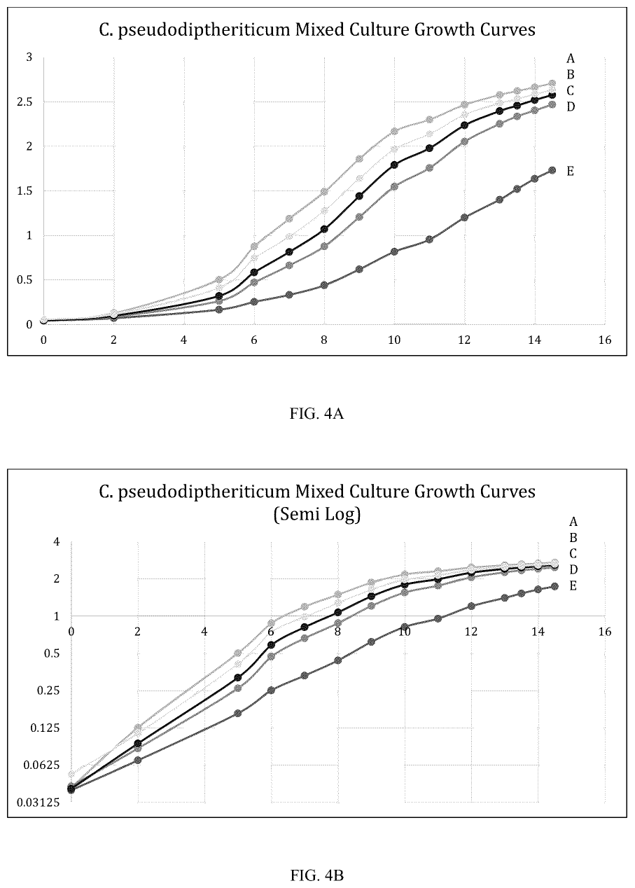

To assay co-culture growth, starting inoculum was varied to achieve mixed cultures while maintaining starting OD600 of 0.04. For example, diluting overnight culture to 0.04 for 400 uL for a 75% JCM 1320 25% ATCC 10700 mix, required 300 uL from the JCM 1320 culture and 10⁰ uL from the ATCC 10700 culture. OD600 measurements were taken over a 14.5 hour time period. Results of OD600 measurements for growth are shown in in FIG. 4A, where samples A, B, C, D, and E are 100% ATCC 10700, 25% JCM 1320 and 75% ATCC 10700, 50% JCM 1320 and 50% ATCC 10700, 75% JCM 1320 and 25% ATCC 10700, and 10⁰% JCM 1320, respectively. Results of Log (OD600) measurements for growth are shown in in FIG. 4B, with the same sample order ordering of data trendlines as in FIG. 4A. Doubling times were as follows: 100% ATCC 10700 [86 minutes], 25% JCM 1320 and 75% ATCC 10700 [89 minutes], 50% JCM 1320 and 50% ATCC 10700 [91 minutes], 75% JCM 1320 and 25% ATCC 10700 [98 minutes], and 100% JCM 1320 [129 minutes]. Doubling time appeared primarily driven by the ATCC 10700 strain. Final stationary phase cell density of mixed cultures was within similar range (OD₆₀₀±0.1).

### Example 6: Consortia Growth Assays

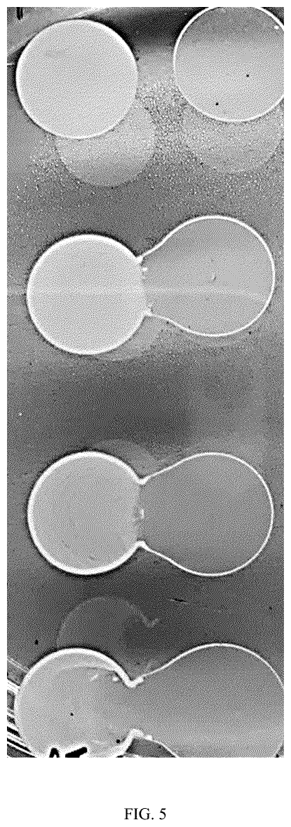

The following was performed to assay co-culture attributes for strains of C. pseudodiptheriticum. First, a direct co-culture of C. pseudodiptheriticum strains ATCC 10700 and JCM 1320 was performed. Both C. pseudodiptheriticum strains were spotted in close proximity at the same time on a Columbia 1% Tween agar plate: four spottings of ATCC 10700 on the left, and for spottings of JCM 1320 on the right. An image capture was taken after 24 hours from spotting, as shown in FIG. 5. As can be seen in FIG. 5, the cultures do not display features of antagonism between the different strains. This is an unexpected result, as co-culturing of C. pseudodiptheriticum clinical isolates has not been reported to date.

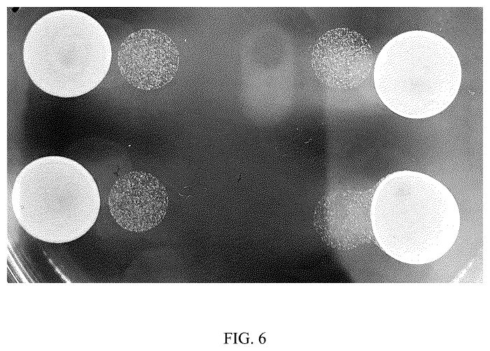

Second, a direct co-culture of C. pseudodiptheriticum strain ATCC 10700 or JCM 1320, and D. pigrum was performed. An image capture was taken after 24 hours from spotting, as shown in FIG. 6. Referring to FIG. 6, on the left is a column after two spottings of ATCC 10700 and just to the right of that is a column after two spottings of D. pigrum. On the right of the image is a column after two spottings of D. pigrum and just to the right of that is a column after two spottings of JCM 1320. As can be seen in FIG. 6, the cultures do not display features of antagonism between the different strains.

While some embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treatment of chronic rhinosinusitis (CRS), the method comprising:
administering to a subject a live, purified population of bacteria, wherein the live, purified population of bacteria comprises:
a plurality of strains of Dolosigranulum pigrum,
wherein the live, purified population of bacteria is present in an amount sufficient for treatment of CRS.

2. The method of claim 1, wherein the plurality of strains of Dolosigranulum pigrum comprises strains comprising different bactericidal mechanisms to pathogenic bacteria.

3. The method of claim 2, wherein the pathogenic bacteria comprises S. aureus, Streptococcus pneumoniae, Pseudomonas aeruginosa, Burkholderia pseudomallei, Haemophilus influenzae, or Moraxella catarrhalis.

4. The method of claim 1, wherein the CRS is with nasal polyps (CRSwNP).

5. The method of claim 1, wherein the CRS is without nasal polyps (CRSsNP).

6. The method of claim 1, wherein the live, purified population of bacteria is present in an amount sufficient for reduction in colonization of a pathogenic bacteria in a nasal cavity of the subject.

7. The method of claim 1, wherein the live, purified population of bacteria is present in an amount sufficient for improvement of sense of smell in the subject.

8. The method of claim 1, wherein the plurality of strains of Dolosigranulum pigrum comprises 1, 2, 3, 4, 5 or more strains listed in Table 2.

9. The method of claim 1, wherein the live, purified population of bacteria further comprises at least one strain of Corynebacterium.

10. The method of claim 1, wherein the live, purified population of bacteria further comprises a plurality of strains of Corynebacterium.

11. The method of claim 9, wherein the at least one strain of Corynebacterium comprises at least on strain of C. accolens, C. pseudodiphtheriticum, C. amycolatum, C. propinquum, C. glutamicum, or C. striatum.

12. The method of claim 9, wherein the at least one strain of Corynebacterium comprises at least on strain of Corynebacterium pseudodiphtheriticum.

13. The method of claim 12, wherein the at least on strain of Corynebacterium pseudodiphtheriticum comprises JCM 1320 and/or ATCC 10700.

14. The method of claim 1, wherein the live, purified population of bacteria is administered intranasally.

15. The method of claim 1, wherein the subject is an infant, a child, or an adult.

16. The method of claim 1, wherein the live, purified population of bacteria is present in a total amount of up to 10\hat{ }15 cfu.

17. The method of claim 1, wherein the live, purified population of bacteria is present in a total amount of at least 10\hat{ }3 cfu, optionally 10\hat{ }3 to 10\hat{ }12 cfu.

18. The method of claim 1, wherein the subject has Cystic Fibrosis.

19. The method of claim 1, wherein the administering is subsequent to the subject receiving endoscopic sinus surgery.

20. The method of claim 1, wherein the subject has a S. aureus infection.

21. The method of claim 1, wherein the subject has an at least 12 week history of CRS, an endoscopic evidence of CRS, a polypoid tissue Grade 1 or less, or any combination thereof.

22. The method of claim 1, wherein the live, purified population of bacteria is in a liquid solution.

23. The method of claim 1, wherein the administration is subsequent to the subject receiving antibiotic and/or steroid therapy.

24. A method for treatment of chronic rhinosinusitis (CRS) without nasal polyps (CRSsNP), the method comprising:
intranasally administering to a subject a live, purified population of bacteria, wherein the live, purified population of bacteria comprises:
at a plurality of strains of Dolosigranulum pigrum comprising different bactericidal mechanisms to pathogenic bacteria; and
at least one strain of Corynebacterium pseudodiphtheriticum,
wherein the live, purified population of bacteria is present in an amount sufficient for treatment of CRSsNP.

25. The method of claim 24, wherein the plurality of strains of D. pigrum comprises at least 3 strains of D. pigrum.

26. The method of claim 24, wherein the plurality of strains of D. pigrum comprises 4 strains of D. pigrum.

27. The method of claim 24, wherein the pathogenic bacteria comprises S. aureus, Streptococcus pneumoniae, Pseudomonas aeruginosa, Burkholderia pseudomallei, Haemophilus influenzae, or Moraxella catarrhalis.
